SEQUENCE LISTING (35 sequences)

SEQ ID NO: 1 - DNA, 294 bp, Streptococcus pneumoniae
atggcaatcgtttcagcagaaaaatttgtccaagcagcccgtgacaacggttatgcagttggtggatttaacacaaacaaccttgagtggactcaagctatcttgcgcgcagcagaagctaaaaaagctccagttttgatccaaacttcaatgggtgctgctaaatacatgggtggttacaaagttgctcgcaacttgatcgctaaccttgttgaatcaatgggtatcactgtaccagtagctatccaccttgaccacggtcactacgaagatgcacttgagtgtatcgaagtt

SEQ ID NO: 2 - DNA, 24 bp, Artificial Sequence
  PRIMER
ggtaccatggcaatcgtttcagca

SEQ ID NO: 3 - DNA, 27 bp, Artificial Sequence
  PRIMER
gagctcaccaacttcgatacactcaag

SEQ ID NO: 4 - protein, 577 aa, Streptococcus pneumoniae
MTEMLKGIAASDGVAVAKAYLLVQPDLSFETITVEDTNAEEARLDAALQASQDELSVIREKAVGTLGEEAAQVFDAHLMVLADPEMISQIKETIRAKKVNAEAGLKEVTDMFITIFEGMEDNPYMQERAADIRDVTKRVLANLLGKKLPNPASINEEVIVIAHDLTPSDTAQLDKNFVKAFVTNIGGRTSHSAIMARTLEIAAVLGTNNITEIVKDGDILAVNGITGEVIINPTDEQAAEFKAAGEAYAKQKAEWALLKDAQTVTADGKHFELAANIGTPKDVEGVNNNGAEAVGLYRTEFLYMDSQDFPTEDEQYEAYKAVLEGMNGKPVVVRTMDIGGDKELPYFDMPHEMNPFLGFRALRISISETGDAMFRTQIRALLRASVHGQLRIMFPMVALLKEFRAAKAVFDEEKANLLAEGVAVADNIQVGIMIEIPAAAMLADQFAKEVDFFSIGTNDLIQYTMAADRMNEQVSYLYQPYNPSILRLINNVIKAAHAEGKWAGMCGEMAGDQQAVPLLVGMGLDEFSMSATSVLRTRSLMKKLDTAKMEEYANRALTECSTMEEVLELQKEYVNFD

SEQ ID NO: 5 - protein, 450 aa, Streptococcus pneumoniae
MGKYFGTDGVRGEANLELTPELAFKLGRFGGYVLSQHETEAPKVFVGRDTRISGEMLESALVAGLLSVGIHVYKLGVLATPAVAYLVETEGASAGVMISASHNPALDNGIKFFGGDGFKLDDEKEAEIEALLDAEEDTLPRPSAEGLGILVDYPEGLRKYEGYLVSTGTPLDGMKVALDTANGAASTSARQIFADLGAQLTVIGETPDGLNINLNVGSTHPEALQEVVKESGSAIGLAFDGDSDRLIAVDENGDIVDGDKIMYIIGKYLSEKGQLAQNTIVTTVMSNLGFHKALNREGINKAVTAVGDRYVVEEMRKSGYNLGGEQSGHVILMDYNTTGDGQLSAVQLTKIMKETGKSLSELAAEVTIYPQKLVNIRVENVMKEKAMEVPAIKAIIEKMEEEMAGNGRILVRPSGTEPLLRVMAEAPTTEEVNYYVDTITDVVRAEIGID

SEQ ID NO: 6 - protein, 420 aa, Streptococcus pneumoniae
MSVSFENKETNRGVLTFTISQDQIKPELDRVFKSVKKSLNVPGFRKGHLPRPIFDQKFGEEALYQDAMNALLPNAYEAAVKEAGLEVVAQPKIDVTSMEKGQDWVITAEVVTKPEVKLGDYKNLEVSVDVEKEVTDADVEERIERERNNLAELVIKEAAAENGDTVVIDFVGSIDGVEFDGGKGENFSLGLGSGQFIPGFEDQLVGHSAGETVDVIVTFPEDYQAEDLAGKEAKFVTTIHEVKAKEVPALDDELAKDIDEEVETLADLKEKYSKELAAAKEEAYKDAVEGAAIDTAVENAEIVELPEEMIHEEVHRSVNEFLGNLQRQGINPDMYFQITGTTQEDLHNQYQAEAESRTKTNLVIEAVAKAEGFDASEEEIQKEVEQLAADYNMEVAQVQNLLSADMLKHDITIKKAVELI

SEQ ID NO: 7 - protein, 693 aa, Streptococcus pneumoniae
MAREFSLEKTRNIGIMAHVDAGKTTTTERILYYTGKIHKIGETHEGASQMDWMEQEQERGITITSAATTAQWNNHRVNIIDTPGHVDFTIEVQRSLRVLDGAVTVLDSQSGVEPQTETVWRQATEYGVPRIVFANKMDKIGADFLYSVSTLHDRLQANAHPIQLPIGSEDDFRGIIDLIKMKAEIYTNDLGTDILEEDIPAEYLDQAQEYREKLIEAVAETDEELMMKYLEGEEITNEELKAGIRKATINVEFFPVLCGSAFKNKGVQLMLDAVIDYLPSPLDIPAIKGINPDTDAEEIRPASDEEPFAALAFKIMTDPFVGRLTFFRVYSGVLQSGSYVLNTSKGKRERIGRILQMHANSRQEIDTVYSGDIAAAVGLKDTTTGDSLTDEKAKIILESINVPEPVIQLMVEPKSKADQDKMGIALQKLAEEDPTFRVETNVETGETVISGMGELHLDVLVDRMRREFKVEANVGAPQVSYRETFRASTQARGFFKRQSGGKGQFGDVWIEFTPNEEGKGFEFENAIVGGVVPREFIPAVEKGLVESMANGVLAGYPMVDVKAKLYDGSYHDVDSSETAFKIAASLSLKEAAKSAQPAILEPMMLVTITVPEENLGDVMGHVTARRGRVDGMEAHGNSQIVRAYVPLAEMFGYATVLRSASQGRGTFMMVFDHYEDVPKSVQEEIIKKNKGED

SEQ ID NO: 8 - protein, 459 aa, Streptococcus pneumoniae
MSKIVVVGANHAGTACINTMLDNFGNENEIVVFDQNSNISFLGCGMALWIGEQIDGAEGLFYSDKEKLEAKGAKVYMNSPVLSIDYDNKVVTAEVEGKEHKESYEKLIFATGSTPILPPIEGVEIVKGNREFKATLENVQFVKLYQNAEEVINKLSDKSQHLDRIAVVGGGYIGVELAEAFERLGKEVVLVDIVDTVLNGYYDKDFTQMMAKNLEDHNIRLALGQTVKAIEGDGKVERLITDKESFDVDMVILAVGFRPNTALAGGKIELFRNGAFLVDKKQETSIPDVYAVGDCATVYDNARKDTSYIALASNAVRTGIVGAYNACGHELEGIGVQGSNGISIYGLHMVSTGLTLEKAKAAGYNATETGFNDLQKPEFMKHDNHEVAIKIVFDKDSREILGAQMVSHDIAISMGIHMFSLAIQEHVTIDKLALTDLFFLPHFNKPYNYITMAALTAEK

SEQ ID NO: 9 - protein, 100 aa, Streptococcus pneumoniae
MKITQEEVTHVANLSKLRFSEEETAAFATTLSKIVDMVELLGEVDTTGVAPTTTMADRKTVLRPDVAEEGIDRDRLFKNVPEKDNYYIKVPAILDNGGDA

SEQ ID NO: 10 - protein, 419 aa, Streptococcus pneumoniae
MTFSFDTAAAQGAVIKVIGVGGGGGNAINRMVDEGVTGVEFIAANTDVQALSSTKAETVIQLGPKLTRGLGAGGQPEVGRKAAEESEETLTEAISGADMVFITAGMGGGSGTGAAPVIARIAKDLGALTVGVVTRPFGFEGSKRGQFAVEGINQLREHVDTLLIISNNNLLEIVDKKTPLLEALSEADNVLRQGVQGITDLITNPGLINLDFADVKTVMANKGNALMGIGIGSGEERVVEAARKAIYSPLLETTIDGAEDVIVNVTGGLDLTLIEAEEASQIVNQAAGQGVNIWLGTSIDESMRDEIRVTVVATGVRQDRVEKVVAPQARSATNYRETVKPAHSHGFDRHFDMAETVELPKQNPRRLEPTQASAFGDWDLRRESIVRTTDSVVSPVERFEAPISQDEDELDTPPFFKNR

SEQ ID NO: 11 - protein, 328 aa, Streptococcus pneumoniae
MTSTKQHKKVILVGDGAVGSSYAFALVNQGIAQELGIIEIPQLHEKAVGDALDLSHALAFTSPKKIYAAQYSDCADADLVVITAGAPQKPGETRLDLVGKNLAINKSIVTQVVESGFKGIFLVAANPVDVLTYSTWKFSGFPKERVIGSGTSLDSARFRQALAEKLDVDARSVHAYIMGEHGDSEFAVWSHANIAGVNLEEFLKDTQNVQEAELIELFEGVRDAAYTIINKKGATYYGIAVALARITKAILDDENAVLPLSVFQEGQYGVENVFIGQPAVVGAHGIVRPVNIPLNDAETQKMQASAKELQAIIDEAWKNPEFQEASKN

SEQ ID NO: 12 - protein, 335 aa, Streptococcus pneumoniae
MVVKVGINGFGRIGRLAFRRIQNVEGVEVTRINDLTDPVMLAHLLKYDTTQGRFDGTVEVKEGGFEVNGKFIKVSAERDPEQIDWATDGVEIVLEATGFFAKKEAAEKHLKGGAKKVVITAPGGNDVKTVVFNTNHDVLDGTETVISGASCTTNCLAPMAKALQDNFGVVEGLMTTIHAYTGDQMILDGPHRGGDLRRARAGAANIVPNSTGAAKAIGLVIPELNGKLDGSAQRVPTPTGSVTELVAVLEKNVTVDEVNAAMKAASNESYGYTEDPIVSSDIVGMSYGSLFDATQTKVLDVDGKQLVKVVSWYDNEMSYTAQLVRTLEYFAKIAK

SEQ ID NO: 13 - protein, 293 aa, Streptococcus pneumoniae
MAIVSAEKFVQAARDNGYAVGGFNTNNLEWTQAILRAAEAKKAPVLIQTSMGAAKYMGGYKVARNLIANLVESMGITVPVAIHLDHGHYEDALECIEVGYTSIMFDGSHLPVEENLKLAKEVVEKAHAKGISVEAEVGTIGGEEDGIIGKGELAPIEDAKAMVETGIDFLAAGIGNIHGPYPVNWEGLDLDHLQKLTEALPGFPIVLHGGSGIPDEQIQAAIKLGVAKVNVNTECQIAFANATRKFARDYEANEAEYDKKKLFDPRKFLADGVKAIQASVEERIDVFGSEGKA

SEQ ID NO: 14 - protein, 336 aa, Streptococcus pneumoniae
MAILVTGGAGYIGSHTVVELLNLGKEVIIVDNLSNSSILVLDRIEAITGIRPVFYELDVCDKQALRKVFEQESIDAAIHFAGYKAVGESVQKPVMYYKNNIMSTLALVEVMSEFNVKKIVFSSSATVYGINNQSPLIETMQTSATNPYGYTKVMLEQILKDVHVADSEWSIALLRYFNPIGAHESGLIGEDPSGIPNNLMPYIAQVAVGKLSELSVFGNDYDTLDGTGVRDYIHVVDLAIGHIKALEKVSEKTDVYIYNLGSGEGTSVLQLVNTFESVNKIPIPYKIVPRRSGDVATCYANADKAYKELNWRTTKSIEDMCRDTWNWQSKNPNGYN

SEQ ID NO: 15 - protein, 620 aa, Streptococcus pneumoniae
MNIIEEIMTKLREDIRNIAIIAHVDHGKTTLVDELLKQSETLDARTELAERAMDSNDIEKERGITILAKNTAVAYNGTRINIMDTPGHADFGGEVERIMKMVDGVVLVVDAYEGTMPQTRFVLKKALEQDLVPIVVVNKIDKPSARPAEVVDEVLELFIELGADDDQLDFPVVYASAINGTSSLSDDPADQEATMAPIFDTIIDHIPAPVDNSDEPLQFQVSLLDYNDFVGRIGIGRVFRGTVKVGDQVTLSKLDGTTKNFRVTKLFGFFGLERREIQEAKAGDLIAVSGMEDIFVGETITPTDAVEALPILHIDEPTLQMTFLVNNSPFAGKEGKWVTSRKVEERLQAELQTDVSLRVDPTDSPDKWTVSGRGELHLSILIETMRREGYELQVSRPEVIVKEIDGVKCEPFERVQIDTPEEYQGSVIQSLSERKGEMLDMISTGNGQTRLVFLVPARGLIGYSTEFLSMTRGYGIMNHTFDQYLPLIPGEIGGRHRGALVSIDAGKATTYSIMSIEERGTIFVNPGTEVYEGMIIGENSRENDLTVNITKAKQMTNVRSATKDQTAVIKTPRILTLEESLEFLNDDEYMEVTPESIRLRKQILNKAEREKANKKKKSAE

SEQ ID NO: 16 - protein, 520 aa, Streptococcus pneumoniae
MSNISTDLQDVEKIIVLDYGSQYNQLISRRIREIGVFSELKSHKISAAEVREVNPVGIILSGGPNSVYEDGSFDIDPEIFELGIPILGICYGMQLLTHKLGGKVVPAGDAGNREYGQSTLTHTPSALFESTPDEQTVLMSHGDAVTEIPADFVRTGTSADCPYAAIENPDKHIYGIQFHPEVRHSVYGNDILRNFALNICKAKGDWSMDNFIDMQIKKIRETVGDKRVLLGLSGGVDSSVVGVLLQKAIGDQLICIFVDHGLLRKGEADQVMDMLGGKFGLNIVKADAAKRFLDKLAGVSDPEQKRKIIGNEFVYVFDDEASKLKDVKFLAQGTLYTDVIESGTDTAQTIKSHHNVGGLPEDMQFELIEPLNTLYKDEVRALGTELGMPDHIVWRQPFPGPGLAIRVMGEITEEKLETVRESDAILREEIAKAGLDRDIWQYFTVNTGVRSVGVMGDGRTYDYTIAIRAITSIDGMTADFAKIPWEVLQKISVRIVNEVDHVNRIVYDITSKPPATVEWE

SEQ ID NO: 17 - protein, 486 aa, Streptococcus pneumoniae
MSKDIRVRYAPSPTGLLHIGNARTALFNYLYARHHGGTFLIRIEDTDRKRHVEDGERSQLENLRWLGMDWDESPESHENYRQSERLDLYQKYIDQLLAEGKAYKSYVTEEELAAERERQEVAGETPRYINEYLGMSEEEKAAYIAEREAAGIIPTVRLAVNESGIYKWHDMVKGDIEFEGGNIGGDWVIQKKDGYPTYNFAVVIDDHDMQISHVIRGDDHIANTPKQLMVYEALGWEAPEFGHMTLIINSETGKKLSKRDTNTLQFIEDYRKKGYLPEAVFNFIALLGWNPGGEDEIFSREEFIKLFDENRLSKSPAAFDQKKLDWMSNDYIKNADLETIFEMAKPFLEEAGRLTDKAEKLVELYKPQMKSVDEIIPLTDLFFSDFPELTEAEREVMTGETVPTVLEAFKAKLEAMTDDEFVTENIFPQIKAVQKETGIKGKNLFMPIRIAVSGEMHGPELPDTIFLLGREKSIQHIENMLKEISK

SEQ ID NO: 18 - protein, 448 aa, Streptococcus pneumoniae
MTSAKEYIQSVFETVKARNGHEAEFLQAVEEFFNTLEPVFEKHPEYIEENILARITEPERVVSFRVPWVDRDGKIQVNRGYRVQFNSAVGPYKGGLRFHPTVNQGILKFLGFEQIFKNVLTGLPIGGGKGGSDFDPKGKTDAEVMRFCQSFMTELQKHIGPSLDVPAGDIGVGGREIGYLYGQYKRLNQFDAGVLTGKPLGFGGSLIRPEATGYGLVYYTEEMLKANGNSFAGKKVVISGSGNVAQYALQKATELGATVISVSDSNGYVIDENGIDFDLLVDVKEKRRARLTEYAAEKATATYHEGTVWTYAGNYDIALPCATQNEINGEAAKRLVAQGVICVSEGANMPSDLDAIKVYKENGIFYGPAKAANAGGVAVSALEMSQNSLRLSWTREEVDGRLKDIMTNIFNTAKTTSETYGLDKDYLAGANIAAFENVANAMIAQGIV

SEQ ID NO: 19 - protein, 346 aa, Streptococcus pneumoniae
MAEITAKLVKELREKSGAGVMDAKKALVETDGDIEKAIELLREKGMAKAAKKADRVAAEGLTGVYVNGNVAAVIEVNAETDFVAKNAQFVELVNTTAKVIAEGKPANNEEALALIMPSGETLEAAYVSATATIGEKISFRRFALIEKTDAQHFGAYQHNGGRIGVISVVEGGDEALAKQLSMHIAAMKPTVLSYKELDEQFVKDELAQLNHVIDQDNESRAMVNKPALPHLKYGSKAQLTDDVIAQAEADIKAELAAEGKPEKIWDKIIPGKMDRFMLDNTKVDQAYTLLAQVYIMDDSKTVEAYLESVNASVVEFARFEVGEGIEKAANDFEAEVAATMAAALNN

SEQ ID NO: 20 - protein, 398 aa, Streptococcus pneumoniae
MAKLTVKDVDLKGKKVLVRVDFNVPLKDGVITNDNRITAALPTIKYIIEQGGRAILFSHLGRVKEEADKAGKSLAPVAADLAAKLGQDVVFPGVTRGAELEAAINALEDGQVLLVENTRYEDVDGKKESKNDPELGKYWASLGDGIFVNDAFGTAHRAHASNVGISANVEKAVAGFLLENEIAYIQEAVETPERPFVAILGGSKVSDKIGVIENLLEKADNVLIGGGMTYTFYKAQGIEIGNSLVEEDKLDVAKALLEKANGKLILPVDSKEANAFAGYTEVRDTEGEAVSEGFLGLDIGPKSIAKFDEALTGAKTVVWNGPMGVFENPDFQAGTIGVMDAIVKQPGVKSIIGGGDSAAAAINLGRADKFSWISTGGGASMELLEGKVLPGLAALTEK

SEQ ID NO: 21 - protein, 400 aa, Streptococcus pneumoniae
MNEFEDLLNSVSQVETGDVVSAEVLTVDATQANVAISGTGVEGVLTLRELTNDRDADINDFVKVGEVLDVLVLRQVVGKDTDTVTYLVSKKRLEARKAWDKLVGREEEVVTVKGTRAVKGGLSVEFEGVRGFIPASMLDTRFVRNAERFVGQEFDTKIKEVNAKENRFILSRREVVEAATAAARAEVFGKLAVGDVVTGKVARITSFGAFVDLGGVDGLVHLTELSHERNVSPKSVVTVGEEIEVKILDLNEEEGRVSLSLKATVPGPWDGVEQKLAKGDVVEGTVKRLTDFGAFVEVLPGIDGLVHVSQISHKRIENPKEALKVGQEVQVKVLEVNADAERVSLSIKALEERPAQEEGQKEEKRAARPRRPRRQEKRDFELPETQTGFSMADLFGDIEL

SEQ ID NO: 22 - protein, 474 aa, Streptococcus pneumoniae
MTKANFGVVGMAVMGRNLALNIESRGYTVAIYNRSKEKTEDVIACHPEKNFVPSYDVESFVNSIEKPRRIMLMVQAGPGTDATIQALLPHLDKGDILIDGGNTFYKDTIRRNEELANSGINFIGTGVSGGEKGALEGPSIMPGGQKEAYELVADVLEEISAKAPEDGKPCVTYIGPDGAGHYVKMVHNGIEYGDMQLIAESYDLMQHLLGLSAEDMAEIFTEWNKGELDSYLIEITADILSRKDDEGQDGPIVDYILDAAGNKGTGKWTSQSSLDLGVPLSLITESVFARYISTYKEERVHASKVLPKPAAFNFEGDKAELIEKIRQALYFSKIISYAQGFAQLRVASKENNWNLPFADIASIWRDGCIIRSRFLQKITDAYNRDADLANLLLDEYFLDVTAKYQQAVRDIVALAVQAGVPVPTFSAAITYFDSYRSADLPANLIQAQRDYFGAHTYQRKDKEGTFHYSWYDEK

SEQ ID NO: 23 - protein, 444 aa, Streptococcus pneumoniae
MNAIQESFTDKLFANYEANVKYQAIENAASHNGIFAALERRQSHVDNTPVFSLDLTKDKVTNQKASGRCWMFAALNTFRHKLISQYKLENFELSQAHTFFWDKYEKSNWFLEQVIATSDQELTSRKVSFLLQTPQQDGGQWDMVVSLFEKYGVVPKSVYPESVSSSSSRELNAILNKLLRQDAQILRDLLVSGADQATVQAKKEDLLQEIFNFLAMSLGLPPRKFDFAYRDKDNNYKSEKGITPQEFYKKYVNLPLEDYVSVINAPTADKPYGKSYTVEMLGNVVGSRAVRYINVPMERLKELAIAQMQAGETVWFGSDVGQLSNRKAGILATDVYDFESSMDIKLTQDKAGRLDYSESLMTHAMVLTGVDLDENGKSTKWKVENSWGDKVGTDGYFVASDAWMDEYTYQIVVRKELLTAEEQAAYGAEPIVLAPWDPMGALAE

SEQ ID NO: 24 - protein, 1058 aa, Streptococcus pneumoniae
MPKRTDIQKIMVIGSGPIIIGQAAEFDYAGTQACLSLKEEGYEVVLVNSNPATIMTDKEIADKVYIEPITLEFVTRILRKEGPDALLPTLGGQTGLNMAMELSKNGILDELGVELLGTKLSAIDQAEDRDLFKQLMEELEQPIPESEIVNTVEEAVAFAATIGYPVIVRPAFTLGGTGGGMCANEKELREITENGLKLSPVTQCLIERSIAGFKEIEYEVMRDSADNALVVCNMENFDPVGIHTGDSIVFAPAQTMSDYENQMLRDASLSIIRALKIEGGCNVQLALDPNSFKYYVIEVNPRVSRSSALASKATGYPIAKLAAKIAVGLTLDEVINPVTGSTYAMFEPALDYVVAKIPRFPFDKFEKGERRLGTQMKATGEVMAIGRNIEESLLKACRSLEIGVHHNEIPELAAVSDDALIEKVVKAQDDRLFYVSEAIRRGYTPEEIAELTKIDIFYLDKLLHIFEIEQELGAHPQDLEVLKTAKLNGFSDRKIAELWGTTDDKVRQLRLENKIVPVYKMVDTCAAEFDSETPYFYSTYGWENESIRSDKESVLVLGSGPIRIGQGVEFDYATVHSVKAIQAAGYEAIIMNSNPETVSTDFSVSDKLYFEPLTFEDVMNVIDLEQPKGVIVQFGGQTAINLAEPLAKAGVTILGTQVADLDRAEDRDLFEQALKELDIPQPPGQTATNEEEAALAARKIGFPVLVRPSYVLGGRAMEIVENEEDLRSYMRTAVKASPDHPVLVDSYIVGQECEVDAISDGKNVLIPGIMEHIERAGVHSGDSMAVYPPQTLSQKVQETIADYTKRLAIGLHCLGMMNIQFVIKDEKVYVIEVNPRASRTVPFLSKVTNIPMAQVATKLILGQSLSELGYQNGLYPESTRVHIKAPVFSFTKLAKVDSLLGPEMKSTGEVMGSDATLEKALYKAFEASYLHLPTFGNVVFTIADDAKEEALNLARRFQNIGYGILATEGTAAFFASHGLQAQPVGKIGDDDKDIPSFVRKGRIQAIINTVGTKRTADEDGEQIRRSAIEHGVPLFTALDTANAMLKVLESRSFVTEAI

SEQ ID NO: 25 - protein, 332 aa, Streptococcus pneumoniae
MTIMSIGIIIASHGEFAAGIHQSGSMIFGEQEKVQVVTFMPNEGPDDLYAKFNNAVAAFDAEDEVLVLADLWSGSPFNQASRVMGENPERKFAIITGLNLPMLIQAYTERLMDAAAGVEKVAANIIKEAKDGIKALPEELNPVEEVASAAAAPVAQTAIPEGTVIGDGKLKINLARLDTRLLHGQVATAWTPDSKANRIIVASDNVAKDDLRKELIKQAAPGNVKANVVPIQKLIEISKDPRFGETHALILFETPQDALRAIEGGVPIKTLNVGSMAHSTGKTLVNTVLSMDKEDVATFEKMRDLGVEFDVRKVPNDSKKDLFDLINKANVK

SEQ ID NO: 26 - protein, 259 aa, Streptococcus pneumoniae
MAVISMKQLLEAGVHFGHQTRRWNPKMAKYIFTERNGIHVIDLQQTVKYADQAYDFMRDAAANDAVVLFVGTKKQAADAVAEEAVRSGQYFINHRWLGGTLTNWGTIQKRIARLKEIKRMEEDGTFEVLPKKEVALLNKQRARLEKFLGGIEDMPRIPDVMYVVDPHKEQIAVKEAKKLGIPVVAMVDTNTDPDDIDVIIPANDDAIRAVKLITAKLADAIIEGRQGEDAVAVEAEFAALETQADSIEEIVEVVEGDNA

SEQ ID NO: 27 - protein, 312 aa, Streptococcus pneumoniae
MTTNRLQVSLPGLDLKNPIIPASGCFGFGQEYAKYYDLNLLGSIMIKATTLEPRFGNPTPRVAETPAGMLNAIGLQNPGLEVVLAEKLPWLEREYPNLPIIANVAGFSKQEYAAVSHGISKATNVKAIELNISCPNVDHCNHGLLIGQDPDLAYDVVKAAVEASEVPVYVKLTPSVTDIVTVAKAAEDAGASGLTMINTLVGMRFDLKTRKPILANGTGGMSGPAVFPVALKLIRQVAQTTDLPIIGMGGVDSTEAALEMYLAGASAIGVGTANFTNPYACPDIIENLPKVMDKYGISSLEELRQEVKESLR

SEQ ID NO: 28 - protein, 307 aa, Streptococcus pneumoniae
MSENQQALNHVVSMEDLTVDQVMKLIKRGIEFKNGAQLPYEDHPIVSNLFFEDSTRTHKSFEVAEIKLGLERLDFDVKTSSVNKGETLYDTILTLSALGVDVCVIRHPEVDYYRELIASPTITTSIINGGDGSGQHPSQSLLDLMTIYEEFGHFEGLKVAIAGDLDHSRVAKSNMQILKRLGAELFFAGPEEWRSQEFADYGQFVTIDEIIDQVDVMMFLRVQHERHDSGAVFSKEDYHAQHGLTQERYDRLKETAILMHPAPINRDVEIADHLVEAPKSRIVQQMTNGVFVRMAILESVLASRNAN

SEQ ID NO: 29 - protein, 398 aa, Streptococcus pneumoniae
MAKEKYDRSKPHVNIGTIGHVDHGKTTLTAAITTVLARRLPSSVNQPKDYASIDAAPEERERGITINTAHVEYETEKRHYAHIDAPGHADYVKNMITGAAQMDGAILVVASTDGPMPQTREHILLSRQVGVKHLIVFMNKVDLVDDEELLELVEMEIRDLLSEYDFPGDDLPVIQGSALKALEGDSKYEDIVMELMNTVDEYIPEPERDTDKPLLLPVEDVFSITGRGTVASGRIDRGIVKVNDEIEIVGIKEETQKAVVTGVEMFRKQLDEGLAGDNVGVLLRGVQRDEIERGQVIAKPGSINPHTKFKGEVYILTKEEGGRHTPFFNNYRPQFYFRTTDVTGSIELPAGTEMVMPGDNVTIDVELIHPIAVEQGTTFSIREGGRTVGSGMVTEIEA

SEQ ID NO: 30 - protein, 46 aa, Streptococcus pneumoniae
MKSTKEEIQTIKTLLKDSRTAKYHKRLQIVLFCLMGKSYKEIIELL

SEQ ID NO: 31 - protein, 398 aa, Streptococcus pneumoniae
MAKLTVKDVDLKGKKVLVRVDFNVPLKDGVITNDNRITAALPTIKYIIEQGGRAILFSHLGRVKEESDKAGKSLAPVAADLAAKLGQDVVFPGVTRGAELEAAINALEDGQVLLVENTRYEDVDGKKESKNDPELGKYWASLGDGIFVNDAFGTAHRAHASNVGISANVEKAVAGFLLENEIAYIQEAVETPERPFVAILGGSKVSDKIGVIENLLEKADKVLIGGGMTYTFYKAQGIEIGNSLVEEDKLDVAKALLEKANGKLILPVDSKEANAFAGYTEVRDTEGEAVSEGFLGLDIGPKSIAKFDEALTGAKTVVWNGPMGVFENPDFQAGTIGVMDAIVKQPGVKSIIGGGDSAAAAINLGRADKFSWISTGGGASMELLEGKVLPGLAALTEK

SEQ ID NO: 32 - protein, 276 aa, Streptococcus pneumoniae
MKKIVKYSSLAALALVAAGVLAACSGGAKKEGEAASKKEIIVATNGSPKPFIYEENGELTGYEIEVVRAIFKDSDKYDVKFEKTEWSGVFAGLDADRYNMAVNNLSYTKERAEKYLYAAPIAQNPNVLVVKKDDSSIKSLDDIGGKSTEVVQATTSAKQLEAYNAEHTDNPTILNYTKADLQQIMVRLSDGQFDYKIFDKIGVETVIKNQGLDNLKVIELPSDQQPYVYPLLAQGQDELKSFVDKRIKELYKDGTLEKLSKQFFGDTYLPAEADIK

SEQ ID NO: 33 - protein, 630 aa, Streptococcus pneumoniae
MTRYQDDFYDAINGEWQQTAEIPADKSQTGGFVDLDQEIEDLMLATTDKWLAGEEVPEDAILENFVKYHRLVRDFDKREADGITPVLPLLKEFQELETFADFTAKLAEFELAGKPNFLPFGVSPDFMDARINVLWASAPSTILPDTTYYAEEHPQREELLTLWKESSANLLKAYDFSDEEIEDLLEKRLELDRRVAAVVLSNEESSEYAKLYHPYSYEDFKKFAPALPLDDFFKAVIGQLPDKVIVDEERFWQAAEQFYSEEAWSLLKATLILSVVNLSTSYLTEDIRVLSGAYSRALSGVPEAKDKVKAAYHLAQEPFKQALGLWYAREKFSPEAKADVEKKVATMIDVYKERLLKNDWLTPETCKQAIVKLNVIKPYIGYPEELPARYKDKVVNETASLFENALAFARVEIKHSWSKWNQPVDYKEWGMPAHMVNAYYNPQKNLIVFPAAILQAPFYDLHQSSSANYGGIGAVIAHEISHAFDTNGASFDENGSLKDWWTESDYAAFKEKTQKVIDQFDGQDSYGATINGKLTVSENVADLGGIAAALEAAKREADFSAEEFFYNFGRIWRMKGRPEFMKLLASVDVHAPAKLRVNVQVPNFDDFFTTYDVKEGDGMWRSPEERVIIW

SEQ ID NO: 34 - protein, 149 aa, Streptococcus pneumoniae
MIGVVARENAAEQIKQYQKFTVNISDETSMLAMEQAGFISHQEKLERLGVHYEISERTQIPILDACPLVLDCRVDRIVEEDGICHIFAKILERLVAPELLDEKGHFKNQLFAPTYFMGDGYQRVYRYLDKRVDMKGSFIKKARKKDGKN

SEQ ID NO: 35 - protein, 149 aa, Streptococcus pneumoniae
MIGVVARENAAEQIKQYQKFTVNISDETSMLAMEQAGFISHQEKLERLGVHYEISERTQTPILDACPLVLDCRVDRIVEEDGICHIFAKILERLVAPELLDEKGHFKNQLFAPTYFMGDGYQRVYRYLDKRVDMKGSFIKKARKKDGKN

(19) United States
(12) Patent Application Publication
(10) Pub. No.: US 2009/0252756 A1
(43) Pub. Date: Oct. 8, 2009

(54) PROTEIN-BASED STREPTOCOCCUS PNEUMONIAE VACCINES

(76) Inventor: Yaffa Mizrachi-Nebenzahl, Beer Sheva (IL)

Correspondence Address: WINSTON & STRAWN LLP;PATENT DEPARTMENT, 1700 K STREET, N.W., WASHINGTON, DC, 20006, US

(21) Appl. No.: 12/435,781

(22) Filed: May 5, 2009

Related U.S. Application Data
(63) Continuation-in-part of application No. 12/363,383, filed on Jan. 30, 2009.
Application No. 12/363,383 is a division of application No. 10/953,513, filed on Sep. 30, 2004, now Pat. No. 7,504,110.
Application No. 10/953,513 is a continuation-in-part of application No. PCT/IL03/00271, filed on Apr. 1, 2003.
(60) Provisional application No. 60/368,981, filed on Apr. 2, 2002.

(51) Int. Cl. A61K 39/09

(52) U.S. Cl. 424/190.1; 424/244.1

(57) ABSTRACT

Vaccine compositions and methods for protecting a mammalian subject against infection with S. pneumoniae are disclosed. The vaccines and methods comprise an effective amount of one or more Streptococcus pneumoniae cell wall and/or cell membrane proteins and/or immunogenically-active fragments, derivatives or modifications thereof, wherein said proteins are selected from a defined group of proteins associated with age-dependent immunological responses.

### CROSS-REFERENCE TO RELATED APPLICATIONS

This application is a continuation-in-part of U.S. application Ser. No. 12/363,383, filed Jan. 30, 2009, which in turn is a divisional of U.S. application Ser. No. 10/953,513, filed Sep. 30, 2004, now U.S. Pat. No. 7,504,110, which in turn is a continuation-in-part of International Application No. PCT/IL2003/00027 1, filed Apr. 1, 2003, which claims the benefit of U.S. Provisional Application No. 60/368,981, filed Apr. 2, 2002. The contents of all prior US applications are expressly incorporated herein by reference thereto.

### FIELD OF THE INVENTION

The present invention relates to vaccine compositions and methods for protecting against infection with Streptococcus pneumoniae. More specifically, the present invention provides vaccine compositions comprising S. pneumoniae cell wall or cell membrane proteins associated with an age-dependent immune response.

### BACKGROUND OF THE INVENTION

The Gram-positive bacterium Streptococcus pneumoniae is a major cause of disease, suffering and death worldwide. Diseases caused by infection with this agent include otitis media, pneumonia, bacteremia, sepsis and meningitis. In some cases, infected individuals may become asymptomatic carriers of S. pneumoniae, thereby readily allowing the rapid spread of this infective agent throughout the population. In view of the serious consequences of infection with S. pneumoniae, as well as its rapid spread within and between populations, there is an urgent need for safe, effective vaccination regimes. Current methods of vaccination are based on inoculation of the subject with polysaccharides obtained from the capsules of S. pneumoniae. While these polysaccharide-based vaccine preparations have been found to be reasonably efficacious when used to prevent infection in adult populations, they are significantly less useful in the treatment of young children (under two years of age) and the elderly. One commonly-used capsular polysaccharide 23-valent vaccine, for example, has been found to be only 60% effective in preventing S. pneumoniae invasive disease in elderly subjects and completely incapable of yielding clinically-useful antibody responses in the under-two age group (Shapiro E. D. et al., 1991, N. Engl. J. Med. 325: 1453-1460). In an attempt to increase the immunogenicity of these vaccines, various compositions comprising capsular polysaccharides that have been conjugated with various carrier or adjuvant proteins have been used. Although vaccines of this type constitute an improvement in relation to the un-conjugated polysaccharide vaccines, they have not overcome the problem of coverage, since they are effective against only about 10% of the 92 known capsular serotypes. Consequently, upon vaccination, repopulation with serotypes not present in the vaccine occurs.

In the cases of certain other bacteria of pathogenic importance for human and other mammalian species, vaccines comprising immunogenic virulence proteins are currently being developed. Such protein-based vaccines should be of particular value in the case of vulnerable subjects such as very young children, in view of the fact that such subjects are able to produce antibodies against foreign proteins. Unfortunately, very little is known of the molecular details of the life cycle of S. pneumoniae, or of the nature of role of the various virulence factors which are known or thought to be involved in targeting and infection of susceptible hosts.

Several publications describe and characterize specific S. pneumoniae proteins. For example, U.S. Pat. Nos. 5,958,734, 5,976,840, 6,165,760 and 6,300,119 disclose S. pneumoniae GtS polypeptides of various lengths, polynucleotides encoding them and methods for producing such polypeptides by recombinant techniques. WO 02/077021 discloses the sequences of about 2,500 S. pneumoniae genes and their corresponding amino acid sequences from type 4 strain that were identified in silico. U.S. Pat. No. 6,699,703 and its counterparts disclose about 2600 S. pneumoniae polypeptides and methods for producing such polypeptides by recombinant techniques, compositions comprising same and methods of use in the preparation of a vaccine. WO 98/23631 relates to 111 Streptococcal polynucleotides identified as having a GUG start codon, which encodes a Val residue, to polypeptides encoded by such polynucleotides, and to their production and uses. WO 02/083833 discloses 376 S. pneumoniae polypeptide antigens which are surface localized, membrane associated, secreted or exposed on the bacteria, for preparation of a diagnostic kit and or vaccine. Although suggested in part of the publications, no working examples for the use of the proteins as antigens in the production of a vaccine were provided. Furthermore, none of these references disclose or suggest that use of selected protein antigens which do no elicit immune response in infants and in elderly, improve the outcome of vaccination against S. pneumoniae.

There is an unmet need to provide protein-based vaccine compositions which overcome the problems and drawbacks of currently available vaccines, by being effective against a wide range of different S. pneumoniae serotypes, and capable of protecting all age groups including infants and elderly.

### SUMMARY OF THE INVENTION

It has now been found that it is possible to protect individuals against infection with S. pneumoniae by means of administering to said individuals a vaccine composition comprising one or more proteins isolated from the outer layers of the aforementioned bacteria and/or one or more immunonologically-active fragments, derivatives or modifications thereof. Unexpectedly, it was found that a defined set of proteins, associated with age-dependent immunity, are effective in vaccine compositions against a wide range of different S. pneumoniae serotypes, and in all age groups, including those age groups that do not produce anti-S. pneumoniae antibodies following vaccination with polysaccharide-based compositions, or those resulting in a shift in serotype distribution towards those pneumococcal capsular polysaccharides that are not present in the vaccine. These age groups include infants aged 0-4 years and elderly. Thus, the use of the set of antigens in accordance with the principle of the invention overcomes the disadvantages of known vaccines.

It is now disclosed that the antibody response to S. pneumoniae proteins increases with age in infants and this increase correlates negatively with morbidity. Antibodies to S. pneumoniae protein antigens develop in humans during the asymptomatic carriage and invasive disease. Infants below two years of age who are at most risk from pneumococcal infections do not respond efficiently to currently available polysaccharide-based vaccination. It is now unexpectedly shown, using sera longitudinally collected from healthy children, exposed to bacterial infections that there is an age-dependent enhancement of the antibody response to certain S. pneumoniae surface protein antigens, while in most other proteins there is no enhancement of immunogenicity during the checked time period. This enhancement, with age, of antibody responses against a set of specific pneumococcal surface proteins is implicated in the development of natural immunity and was used in the present invention to identify candidate antigens (herein “age dependent proteins”) for use in improved vaccine compositions effective in all age groups, including infants, immunocompromized subjects and elderly.

In elderly subjects capsular polysaccharide based vaccines are only 60% effective in preventing S. pneumoniae invasive disease. An elderly subject should be vaccinated at least once in five years and the vaccination efficacy is reduced in each repeated vaccination. The protein-based vaccines of the present invention, which are T-cell dependent antigens, are expected to be more effective than the polysaccharide-based vaccines in elderly subjects.

The present invention provides a method for protecting individuals against infection with S. pneumoniae by the use of a protein-based vaccine.

The present invention further provides a protein-based vaccine that is prepared from at least one of a specific set of immunogenic cell wall and/or cell membrane proteins of S. pneumoniae, having age-dependent immune responses, or from one or more immunologically-active fragments, derivatives or modifications thereof.

According to one aspect of the present invention, a vaccine composition comprises as an active ingredient one or more isolated proteins selected from one or more S. pneumoniae cell wall or cell membrane proteins or immunoglogically-active protein fragments, derivatives or modifications thereof, which are associated with an age-dependent immune response. According to preferred embodiments, this aspect of the invention said the age-dependent S. pneumoniae cell wall and/or cell membrane protein is selected from the group consisting of: phosphoenolpyruvate protein phosphotransferase (Accession No. NP_{—}345645, SEQ ID NO:4); phosphoglucomutase/phosphomannomutase family protein (Accession No. NP_{—}346006, SEQ ID NO:5); trigger factor (Accession No. NP_{—}344923, SEQ ID NO:6); elongation factor G/tetracycline resistance protein (tetO), (Accession No. NP_{—}344811, SEQ ID NO:7); NADH oxidase (Accession No. NP_{—}345923, SEQ ID NO:8); Aspartyl/glutamyl-tRNA amidotransferase subunit C (Accession No. NP_{—}344960, SEQ ID NO:9); cell division protein FtsZ (Accession No. NP_{—}346105, SEQ ID NO:10); L-lactate dehydrogenase (Accession No. NP_{—}345686, SEQ ID NO:11); glyceraldehyde 3-phosphate dehydrogenase (GAPDH), (Accession No. NP_{—}346439, SEQ ID NO:12); fructose-bisphosphate aldolase (Accession No. NP_{—}345117, SEQ ID NO:13); UDP-glucose 4-epimerase (Accession No. NP_{—}346261, SEQ ID NO:14); elongation factor Tu family protein (Accession No. NP_{—}358192, SEQ ID NO:15); Bifunctional GMP synthase/glutamine amidotransferase protein (Accession No. NP_{—}345899, SEQ ID NO:16); glutamyl-tRNA synthetase (Accession No. NP_{—}346492, SEQ ID NO:17); glutamate dehydrogenase (Accession No. NP_{—}345769, SEQ ID NO:18); Elongation factor TS (Accession No. NP_{—}346622, SEQ ID NO:19); phosphoglycerate kinase (TIGR4) (Accession No. AAK74657, SEQ ID NO:20); 30S ribosomal protein S1 (Accession No. NP_{—}345350, SEQ ID NO:21); 6-phosphogluconate dehydrogenase (Accession No. NP_{—}357929, SEQ ID NO:22); aminopeptidase C (Accession No. NP_{—}344819, SEQ ID NO:23); carbamoyl-phosphate synthase (large subunit) (Accession No. NP_{—}345739, SEQ ID NO:24); PTS system, mannose-specific IIAB components (Accession No. NP_{—}344822, SEQ ID NO:25); 30S ribosomal protein S2 (Accession No. NP_{—}346623, SEQ ID NO:26); dihydroorotate dehydrogenase 1B (Accession No. NP_{—}358460, SEQ ID NO:27); aspartate carbamoyltransferase catalytic subunit (Accession No. NP_{—}345741, SEQ ID NO:28); elongation factor Tu (Accession No. NP_{—}345941, SEQ ID NO:29); Pneumococcal surface immunogenic protein A (PsipA) (Accession No. NP_{—}344634, SEQ ID NO:30); phosphoglycerate kinase (R6) (Accession No. NP_{—}358035, SEQ ID NO:31); ABC transporter substrate-binding protein (Accession No. NP_{—}344690, SEQ ID NO:32); endopeptidase O (Accession No. NP_{—}346087, SEQ ID NO:33); Pneumococcal surface immunogenic protein B (PsipB) (Accession No. NP_{—}358083, SEQ ID NO:34); Pneumococcal surface immunogenic protein C (PsipC) (Accession No. NP_{—}345081, SEQ ID NO:35).

According to some embodiments the one or more bacterial proteins of the vaccine are effective in all age groups, including those age groups that do not produce anti-S. pneumoniae antibodies following vaccination with polysaccharide-based vaccines; or exposure to the bacteria.

According to one embodiment the age group comprises infants less than four years of age. According to another embodiment the age group comprises infants less than two years of age.

According to one embodiment the age group comprises elderly subjects.

According to yet another embodiment the age group comprises children older the 4 years of age and adult subjects.

According to another embodiment the age group comprises immunocompromised subjects.

The vaccine compositions of the present invention may also contain other, non-immunologically-specific additives, diluents and excipients. For example, in many cases, the vaccine compositions of the present invention may contain, in addition to the S. pneumoniae cell-wall and/or cell-membrane protein(s), one or more adjuvants.

Pharmaceutically acceptable adjuvants include, but are not limited to water in oil emulsion, lipid emulsion, and liposomes. According to specific embodiments the adjuvant is selected from the group consisting of: MONTANIDE®, alum, muramyl dipeptide, GELVAC®, chitin microparticles, chitosan, cholera toxin subunit B, labile toxin, AS21A, AS02V, INTRALIPID®, Lipofundin, Monophosphoryl lipid A; RIBI: monophosphoryl lipid A with Mycobacterial cell wall components (muramy tri peptide), ISCOMs Immune stimulating complexes, CpG, and DNA vaccines such as pVAC. Also included are immune enhancers such as cytokines.

In some embodiments the vaccine composition is formulated for intramuscular, intranasal, oral, intraperitoneal, subcutaneous, topical, intradermal and transdermal delivery. In some embodiments the vaccine is formulated for intramuscular administration. In other embodiments the vaccine is formulated for oral administration. In yet other embodiments the vaccine is formulated for intranasal administration.

In one particularly preferred embodiment, the method of the present invention for protection of mammalian subjects against infection with S. pneumoniae comprises administering to a subject in need of such protection an effective amount of at least one cell wall and/or cell membrane proteins associated with age-related immune response, and/or immunogenically-active fragments, derivatives or modifications thereof, wherein said at least one protein is selected from the group consisting of: fructose-bisphosphate aldolase (FBA, NP_{—}345117, SEQ ID NO:13), Phosphoenolpyruvate protein phosphotransferase (PPP) NP_{—}345645 (SEQ ID NO:4), Glutamyl tRNA synthetase (GtS, NP_{—}346492, SEQ ID NO:17), NADH oxidase (NOX, NP_{—}345923, SEQ ID NO:8), Pneumococcal surface immunogenic protein B (PsipB; NP_{—}358083, SEQ ID NO:34), trigger factor (TF, NP 344923, SEQ ID NO:6), FtsZ cell division protein (NP_{—}346105, SEQ ID NO:10), PTS system, mannose-specific IIAB components (PTS, NP_{—}344822, SEQ ID NO:25), and Elongation factor G (EFG, NP344811, SEQ ID NO:7).

According to some embodiments at least one protein of the vaccine composition is an enzyme involved in glycolysis. According to a specific embodiment the at least one protein involved in glycolysis is selected from the group consisting of: L-lactate dehydrogenase (SEQ ID NO:11), UDP-glucose 4-epimerase (SEQ ID NO:14), fructose-bisphosphate aldolase (SEQ ID NO:13), glyceraldehyde-3-phosphate dehydrogenase (SEQ ID NO:12), phosphoglycerate kinase (SEQ ID NO:31) and 6-phosphoglutamate dehydrogenase (SEQ ID NO:22).

According to another embodiment at least one protein of the vaccine composition is an enzyme involved in protein synthesis. According to a specific embodiment the protein involved in protein synthesis is glutamyl-tRNA amidotransferase (SEQ ID NO:16) or glutamyl-tRNA synthetase (SEQ ID NO:17).

According to other embodiments at least one protein of the vaccine composition is an enzyme belonging to the other physiological pathways selected from: NADP glutamate dehydrogenase (NP_{—}345769), aminopeptidase C (Accession No. NP_{—}344819, SEQ ID NO:23), carbamoylphosphate synthase (Accession No. NP_{—}345739, SEQ ID NO:24), aspartate carbamoyltransferase (Accession No. NP_{—}345741, SEQ ID NO:28), NADH oxidase (NOX, Accession No. NP_{—}345923, SEQ ID NO:8), Pneumococcal surface immunogenic protein B (PsipB, Accession No. NP_{—}358083, SEQ ID NO:34); and pyruvate oxidase.

In some embodiments the cell wall and/or cell membrane proteins are lectins. According to specific embodiments the lectin proteins are selected from the group consisting of: Fructose-bisphosphate aldolase (NP 345117, SEQ ID NO:13); Aminopeptidase C (NP_{—}344819, SEQ ID NO:23).

According to some embodiments the S. pneumoniae proteins and/or fragments, derivatives or modifications thereof are lectins and the vaccine compositions comprising them are particularly efficacious in the prevention of localized S. pneumoniae infections. In one preferred embodiment, the localized infections are infections of mucosal tissue, particularly of nasal and other respiratory mucosa.

In alternative embodiments of the method of the invention, the cell wall and/or cell membrane proteins are non-lectins.

In specific embodiments the non-lectin proteins are selected from the group consisting of: Phosphomannomutase (NP 346006, SEQ ID NO:5); Trigger factor (NP 344923, SEQ ID NO:6); NADH oxidase (NP 345923, SEQ ID NO:8); L-lactate dehydrogenase (NP 345686, SEQ ID NO:11); Glutamyl-tRNA synthetase (NP 346492, SEQ ID NO:17).

According to other embodiments the S. pneumoniae proteins and/or their fragments, derivatives or modifications used in the aforementioned methods, compositions and vaccines are non-lectins, and the vaccine compositions are particularly efficacious in the prevention of systemic S. pneumoniae infections.

In another preferred embodiment of the method of the invention, vaccine composition comprises at least one lectin protein and at least one non-lectin protein.

The present invention is directed according to another aspect to a method for preventing infection of mammalian subjects with S. pneumoniae, wherein said method comprises administering to a subject in need of such treatment an effective amount of one or more S. pneumoniae cell wall and/or cell membrane proteins associated with age-related immune response, and/or immunogenically-active fragments, derivatives or modifications thereof, wherein said proteins are selected from the group consisting of:

phosphoenolpyruvate protein phosphotransferase (Accession No. NP_{—}345645, SEQ ID NO:4); phosphoglucomutase/phosphomannomutase family protein (Accession No. NP_{—}346006, SEQ ID NO:5); trigger factor (Accession No. NP_{—}344923, SEQ ID NO:6); elongation factor G/tetracycline resistance protein (tetO), (Accession No. NP_{—}344811, SEQ ID NO:7); NADH oxidase (Accession No. NP_{—}345923, SEQ ID NO:8); Aspartyl/glutamyl-tRNA amidotransferase subunit C (Accession No. NP_{—}344960, SEQ ID NO:9); cell division protein FtsZ (Accession No. NP_{—}346105, SEQ ID NO:10); L-lactate dehydrogenase (Accession No. NP_{—}345686, SEQ ID NO:11); glyceraldehyde 3-phosphate dehydrogenase (GAPDH), (Accession No. NP_{—}346439, SEQ ID NO:12); fructose-bisphosphate aldolase (Accession No. NP_{—}345117, SEQ ID NO:13); UDP-glucose 4-epimerase (Accession No. NP_{—}346261, SEQ ID NO:14); elongation factor Tu family protein (Accession No. NP_{—}358192, SEQ ID NO:15); Bifunctional GMP synthase/glutamine amidotransferase protein (Accession No. NP_{—}345899, SEQ ID NO:16); glutamyl-tRNA synthetase (Accession No. NP_{—}346492, SEQ ID NO:17); glutamate dehydrogenase (Accession No. NP_{—}345769, SEQ ID NO:18); Elongation factor TS (Accession No. NP_{—}346622, SEQ ID NO:19); phosphoglycerate kinase (TIGR4) (Accession No. AAK74657, SEQ ID NO:20); 30S ribosomal protein S1 (Accession No. NP_{—}345350, SEQ ID NO:21); 6-phosphogluconate dehydrogenase (Accession No. NP_{—}357929, SEQ ID NO:22); aminopeptidase C (Accession No. NP_{—}344819, SEQ ID NO:23); carbamoyl-phosphate synthase (large subunit) (Accession No. NP_{—}345739, SEQ ID NO:24); PTS system, mannose-specific IIAB components (Accession No. NP_{—}344822, SEQ ID NO:25); 30S ribosomal protein S2 (Accession No. NP_{—}346623, SEQ ID NO:26); dihydroorotate dehydrogenase 1B (Accession No. NP_{—}358460, SEQ ID NO:27); aspartate carbamoyltransferase catalytic subunit (Accession No. NP_{—}345741, SEQ ID NO:28); elongation factor Tu (Accession No. NP_{—}345941, SEQ ID NO:29); Pneumococcal surface immunogenic protein A (PsipA) (Accession No. NP_{—}344634, SEQ ID NO:30); phosphoglycerate kinase (R6) (Accession No. NP_{—}358035, SEQ ID NO:31); ABC transporter substrate-binding protein (Accession No. NP_{—}344690, SEQ ID NO:32); endopeptidase O (Accession No. NP_{—}346087, SEQ ID NO:33); Pneumococcal surface immunogenic protein B (PsipB) (Accession No. NP_{—}358083, SEQ ID NO:34); Pneumococcal surface immunogenic protein C (PsipC) (Accession No. NP_{—}345081, SEQ ID NO:35).

Vaccine compositions of the present invention can be administered to a subject in need thereof, prior to, during or after occurrence of infection or inoculation with S. pneumoniae.

The vaccine compositions of the present invention are administered, according to one embodiment by means of injection. According to some embodiments the injection route is selected from the group consisting of: intramuscular, intradermal or subcutaneous. According to other embodiments the injection route is selected from intravenous and intraperitoneal. According to yet other embodiments the vaccine compositions of the present invention are administered by nasal or oral routes.

According to some embodiments the S. pneumoniae proteins and/or fragments, derivatives or modifications thereof are lectins and the vaccine compositions comprising them are particularly efficacious in the prevention of localized S. pneumoniae infections. In one preferred embodiment, the localized infections are infections of mucosal tissue, particularly of nasal and other respiratory mucosa.

According to other embodiments the S. pneumoniae proteins and/or their fragments, derivatives or modifications used in the aforementioned methods, compositions and vaccines are non-lectins, and the vaccine compositions are particularly efficacious in the prevention of systemic S. pneumoniae infections.

In another preferred embodiment of the method of the invention, vaccine composition comprises at least one lectin protein and at least one non-lectin protein.

In one preferred embodiment of the method of the invention, the mammalian subject is a human subject.

The aforementioned vaccine compositions may clearly be used for preventing infection of the mammalian subjects by S. pneumoniae. However, said vaccine composition is not restricted to this use alone. Rather it may be usefully employed to prevent infection by any infectious agent whose viability or proliferation may be inhibited by the antibodies generated by a host in response to the inoculation therein of the one or more S. pneumoniae proteins provided in said composition.

DNA vaccines comprising at least one polynucleotide sequence encoding age-dependent bacterial proteins according to the invention are also within the scope of the present invention, as well as methods for protecting a mammalian subject against infection with S. pneumoniae comprising administering such polynucleotide sequence to a subject. According to one embodiment the present invention provides a vaccine composition comprising at least one polynucleotide sequence encoding a protein selected from one or more S. pneumoniae cell wall or cell membrane proteins or immunogenically-active protein fragments, derivatives or modifications thereof, which is associated with an age-dependent immune response. According to some embodiments the DNA vaccine composition further comprises at least one polynucleotide sequence encoding an adjuvant peptide or protein. According to a preferred embodiment a DNA vaccine according to the invention is administered by intramuscular injection.

The present invention discloses, according to yet a further aspect, a method for identifying bacterial proteins having age-dependent immunogenicity. Identified age-dependent proteins can be used in vaccine compositions against pathogens expressing said proteins.

According to certain embodiments, a method for identifying a bacterial protein having age-dependent immunogenicity is provided the method comprises the steps of: providing an extract of the cell wall and/or cell membrane of the pathogen; separating the extract by 2D-electrophoresis or micro-chromatography; blotting the protein extract to a matrix; probing the blots with sera collected longitudinally from children at different ages; identifying the protein spots having intensity increasing with age; thereby identifying a protein having age-dependent immunogenicity.

According to some embodiments the protein extract is blotted onto a paper. According to other embodiments the proteins are identified using Matrix Assisted Laser Desorption/Ionization mass spectrometery (MALDI-MS) technique.

According to some embodiments the pathogen is a bacterium. According to specific embodiments the bacterium is S. pneumoniae and the sera are collected from children aged 18, 30 and 42 months. According to another embodiment the pathogen is Streptococcus pyogenes.

All of the above and other characteristics and advantages of the present invention will be further understood from the following illustrative and non-limitative examples of preferred embodiments thereof.

### BRIEF DESCRIPTION OF THE DRAWINGS

FIG. 1 is a photograph of a Western blot in which the sera of mice immunized with (A) recombinant GAPDH and (B) recombinant fructose-bisphosphate aldolase are seen to recognize the corresponding native proteins (CW) (in an electrophoretically-separated total cell wall protein preparation), and the corresponding recombinant protein (R).

FIG. 2 is a photograph of a Western blot in which the sera of mice immunized with pVAC constructs containing the cDNA of S. pneumoniae fructose-bipshosphate aldolase (A) and GAPDH (B) are seen to recognize the corresponding native proteins from electrophoretically-separated total cell wall protein preparation. Sera obtained following immunization with the pVAC parental plasmid did not recognize either of the two proteins (C).

FIG. 3 is a graph describing the ability of recombinant GAPDH and fructose-bisphosphate aldolase to elicit a protective immune response to intraperitoneal and intranasal challenge with a lethal dose of S. pneumoniae in the mouse model system.

FIG. 4 is a photograph of a gel depicting the 297 base pair ALDO 1-containing fragment of S. pneumoniae fructose bisphosphate aldolase.

FIG. 5 depicts an agarose gel separation of ALDO 1 and the pHAT vector after restriction by Kpn1 and SacI enzymes.

FIG. 6 is a photograph of an agarose gel showing the 297 bp PCR amplification product (comprising ALDO 1) obtained from colonies transformed with the pHAT/ALDO 1 construct.

FIG. 7 describes nasopharyngeal and lung colonization in mice following challenge with S. pneumoniae of mice actively immunized with Phosphoenolpyruvate protein phosphotransferase (PPP). FIG. 7A describes colonization three hours following challenge FIG. 7B describes colonization 24 hours following challenge.

FIG. 8 depicts the increased survival of mice following a lethal intranasal inoculation of mice following immunization with recombinant Glutamyl tRNA synthetase (rGtS).

FIG. 9 describes survival of mice following active immunization with recombinant NADH oxidase (rNOX).

FIG. 10 describes survival of mice after passive IP immunization with: anti-rPsipB antiserum, control preimmune serum, or anti-non-lectin protein mixture (NL) serum. The mice were inoculated intraperitonealy with the antiserum 24 and 3 hours prior to bacterial challenge.

FIG. 11 describes active immunization of mice with Trigger factor (TF) using CFA/IFA/IFA immunization protocol in comparison to control (adjuvant) immunized animals.

FIG. 12 describes survival of mice following IP challenge with S. pneumoniae after 1 hour neutralization with anti-FtsZ cell division protein (FtsZ) antiserum, preimmune serum or anti NL serum.

FIG. 13 describes survival of mice following IP challenge with S. pneumoniae neutralized with anti-PTS system, mannose-specific IIAB components (PTS) antiserum, preimmune serum or NL serum.

FIG. 14 describes mice survival after active immunization with Elongation factor G (EFG) with Alum adjuvant in comparison to mice injected with adjuvant only as control.

FIG. 15 reconfirms the age dependent recognition of GtS by sera obtained longitudinally from children attending day care centers and a serum obtained from an adult subject.

FIG. 16 reconfirms the age dependent recognition of NOX, using rNOX, by sera obtained longitudinally from children attending day care centers.

### DETAILED DESCRIPTION OF PREFERRED EMBODIMENTS

As disclosed herein for the first time, specific pneumococcal surface proteins that exhibit age-dependent immunogeicity, which coincide with the development of natural protective immunity. Proteins identified using antibodies against these proteins, present in infant sera, elicit a protective response against S. pneumoniae and can be used for protection against infection with the bacteria. It is now shown that proteins identified as exhibiting age-dependent immune response in infants, or antibodies to such proteins were able to protect mice against infection with S. pneumoniae.

Vaccine compositions according to the present invention may be used for preventing infection of the mammalian subjects by S. pneumoniae. However, said vaccine compositions may be also usefully employed to prevent adhesion of the bacteria to cells and to inhibit and reduce bacterial load and bacterial carriage. It was shown (Daniely et al., 2006, Clin. Exp. Immunol. 144, 254-263; Mizrachi Nebenzahl et al., 2007, J. Infectious Diseases 196:945-53), that antibodies to proteins identified in the present application as possessing age-dependent immunogenicity are capable of inhibiting S. pneumoniae adhesion to human lung cells.

The immunologically variant capsular polysaccharides of S. pneumoniae are used widely for the typing of clinical isolates. There are more than 90 capsular serotypes and their prevalence among human isolates varies with age, disease type and to some extent geographical origin. A 23-valent capsular polysaccharide-based vaccine is licensed for use in adults, but it does not elicit an efficient antibody response or protection in children under 2 years of age and immunocompromised patients. To overcome this lack of responsiveness to the T cell independent polysaccharide antigens in young children the conjugate pneumococcal vaccines were developed. These vaccines consist of 7 to 13 of the most prevalent S. pneumoniae capsular polysaccharides covalently linked to a protein carrier to stimulate T cell responses to the vaccine. These vaccines are highly effective in preventing invasive pneumococcal disease in infants but there are some drawbacks associated with the complexity of the manufacturing process that increase costs and the limited number of various capsular polysaccharides that can be included in the vaccine. Vaccination with conjugate pneumococcal vaccines has recently been shown to result in a shift in serotype distribution toward those pneumococcal capsular polysaccharides that are not present in the vaccine. In addition, geographical variations in the prevalence of clinically important serotypes of S. pneumoniae were described. These concerns combined with the increasing antibiotic resistance are driving research efforts to develop a wide range pneumococcal vaccine that is immunogenic in all age groups and broadly cross-protective against all or most serotypes. In addition proteins are T cell dependent antigen and are more likely to induce long lasting immunological memory.

The reasons to longitudinally start collecting sera from day-care children who are frequently exposed to S. pneumoniae, aiming to identify protein antigens involved in the development of natural immunity to S. pneumoniae, at 18 months of age were:

i. During gestation maternal IgG antibodies cross the placenta and in the initial months of life these maternal antibodies are protecting the infants.

ii. Starting at 6 months of age the levels of the maternal antibodies decline and a gradual increase in the infants' antibodies start to appear.

iii. Children are most susceptible to S. pneumoniae infections between 5-35 months of age. The first decrease in their susceptibility can be observed at between 12-23 months of age however the most significant decrease occurs between 24-35 months of age. It is assumed that natural strong immune response to a protein (for example Pyruvate oxidase and Enolase table 2), preceding this time period is not sufficient to protect children from S. pneumoniae infections. Therefore these proteins which did not elicit natural protection against the bacteria although an immune response against them is high in young infants are not age-dependent.

Immunodeficiencies comprise a highly variable group of diseases. While primary immunodeficiencies result from genetic alteration in genes affecting the immune response, acquired immunodeficiencies result from infection with pathogens that affects the immune system (such as HIV-1). Other conditions that may cause diminution of the immune response and increase susceptibility to infections include malnutrition and diseases such as cancer. Most of the immunocompromised patients have acquired immunodeficiency. Malfunction of the immune system may stem from either lack of or the existence of dysfunctional B cells or T cells or macrophages. In other cases immunodeficiency may result in loss of immune memory cells. Antibody deficiencies comprise the most common types of primary immune deficiencies in human subjects. Such patients are highly susceptible to encapsulated bacterial infections. For example, patients that have B cell immunodeficiencies could benefit from vaccination with the proteins of the present invention, which are T cell dependent antigens. Patients that demonstrated loss of immune memory, including HIV-1 patients, could also benefit from vaccination with the compositions of the present invention.

Thus it was suspected that the most significant development of natural immunity occurs after two years of age and it was chosen to encompass this period in the attempt to identify proteins that the immune responses to them increase with age during this period.

Vaccination of infants in the first year of age with the age-dependent bacterial proteins of the invention is expected to elicit protective immune responses to the bacteria, simulating the development of natural protective immunity that occurs at an older age.

Vaccination protects individuals (and by extension, populations) from the harmful effects of pathogenic agents, such as bacteria, by inducing a specific immunological response to said pathogenic agents in the vaccinated subject.

Vaccines are generally, but not exclusively, administered by means of injection, generally by way of the intramuscular, intradermal or subcutaneous routes. Some vaccines may also be administered by the intravenous, intraperitoneal, nasal or oral routes.

The S. pneumoniae-protein containing preparations of the invention can be administered as either single or multiple doses of an effective amount of said protein. The term “effective amount” is used herein to indicate that the vaccine is administered in an amount sufficient to induce or boost a specific immune response, such that measurable amounts (or an increase in the measurable amounts) of one or more antibodies directed against the S. pneumoniae proteins used may be detected in the serum or plasma of the vaccinated subject. The precise weight of protein or proteins that constitutes an “effective amount” will depend upon many factors including the age, weight and physical condition of the subject to be vaccinated. The precise quantity also depends upon the capacity of the subject's immune system to produce antibodies, and the degree of protection desired. Effective dosages can be readily established by one of ordinary skill in the art through routine trials establishing dose response curves. However, for the purposes of the present invention, effective amounts of the compositions of the invention can vary from 0.01-1,000 μg/ml per dose, more preferably 0.1-500 μg/ml per dose, wherein the usual dose size is 1 ml.

The vaccine compositions of the present invention, capable of protecting subject from infection or inoculation with S. pneumoniae can be administered to a subject in need thereof, prior to, during or after occurrence of infection or inoculation with the bacteria.

In general, the vaccines of the present invention would normally be administered parenterally, by the intramuscular, intravenous, intradermal or subcutaneous routes, either by injection or by a rapid infusion method. Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Besides the abovementioned inert diluents and solvents, the vaccine compositions of the invention can also include adjuvants, wetting agents, emulsifying and suspending agents, or sweetening, flavoring, or perfuming agents.

The vaccines of the present invention will generally comprise an effective amount of one or more S. pneumoniae proteins as the active component, suspended in an appropriate vehicle. In the case of intranasal formulations, for example, said formulations may include vehicles that neither cause irritation to the nasal mucosa nor significantly disturb ciliary function. Diluents such as water, aqueous saline may also be added. The nasal formulations may also contain preservatives including, but not limited to, chlorobutanol and benzalkonium chloride. A surfactant may be present to enhance absorption of the subject proteins by the nasal mucosa. An additional mode of antigen delivery may include an encapsulation technique, which involves complex coacervation of gelatin and chondroitin sulfate (Azhari R, Leong K W. 1991. Complex coacervation of chondroitin sulfate and gelatin and its use for encapsulation and slow release of a model protein. Proc. Symp. Control. Rel. 18: 617; Brown K E, Leong K, Huang C H, Dalal R, Green G D, Haimes H B, Jimenez P A, Bathon J. 1998. Gelatin/chondroitin 6-sulfate microspheres for delivery of therapeutic proteins to the joint. Arthritis Rheum 41: 2185-2195).

### Definitions

The term “immunologically-active” is used herein in ordinary sense to refer to an entity (such as a protein or its fragment or derivative) that is capable of eliciting an immune response when introduced into a host subject.

The term “immunogenic protein” according to the present invention denotes a bacterial protein that was identified by antibodies present in human sera. “Antigenicity” refers to the ability of the bacterial protein to produce antibodies against it in the host. The term “age-related immune response” or “age dependent protein” (as used throughout this application) indicates that the ability of subjects to produce antibodies to the bacterial protein or proteins, causing said immune response, increases with age. In the case of human subjects, said ability is measured over a time scale beginning with neonates and ending at approximately four years of age and adults. In non-human mammalian subjects, the “age-related immune response” is measured over an age range extending from neonates to an age at which the immune system of the young mammal is at a stage of development comparable to that of a pre-puberty human child and adults.

It is to be noted that in the context of the present invention, the terms “fragments”, “derivatives” and “modifications” are to be understood as follows:

“Fragment”: a less than full length portion, or linked portions, of the native sequence of the protein in question, wherein the sequence of said portion is essentially unchanged as compared to the relevant part of the sequence of the native protein. “Derivative”: a full length, and a less than full length portion of the native sequence of the protein in question, wherein either the sequence further comprises (at its termini and/or within said sequence itself) non-native amino acid sequences, i.e. sequences which do not form part of the native protein in question. The term “derivative” also includes within its scope molecular species produced by conjugating chemical groups to the amino residue side chains of the native proteins or fragments thereof, wherein said chemical groups do not form part of the naturally-occurring amino acid residues present in said native proteins. “Modification”: a full length protein or less than full length portion thereof comprising at least one amino acid residue which is not natively present in the same location in the sequence of said protein, which have been introduced as a consequence of mutation of the native sequence (by either random or site-directed processes), by chemical modification or by chemical synthesis.

The term “infection” as used herein in the present application refers to a state in which disease-causing S. pneumoniae have invaded, colonized, spread, adhered, disseminated or multiplied in body cells or tissues. This term encompass the term “inoculation”, namely the state in which the bacteria colonized the nasopharynx but there are no infection symptoms yet.

The term “lectins” is used hereinabove and hereinbelow to indicate proteins having the ability to bind specifically to polysaccharides or oligosaccharides. Conversely, the term “non-lectins” is used to refer to proteins lacking the aforementioned saccharide-binding property, or to proteins which do not bind the saccharides tested in the present application.

### Vaccine Formulation

The vaccines of the present invention comprise at least one bacterial protein exhibiting an age-dependent increase antibody response in infants, fragment, derivative or modification of said bacterial protein, and optionally, an adjuvant. Formulation can contain a variety of additives, such as adjuvant, excipient, stabilizers, buffers, or preservatives. The vaccine can be formulated for administration in one of many different modes.

In preferred embodiment, the vaccine is formulated for parenteral administration, for example intramuscular administration. According to yet another embodiment the administration is orally.

According to yet another embodiment the administration is intradermal. Needles specifically designed to deposit the vaccine intradermally are known in the art as disclosed for example in U.S. Pat.No. 6,843,781 and U.S. Pat. No. 7,250,036 among others. According to other embodiments the administration is performed with a needleless injector.

According to one embodiment of the invention, the vaccine is administered intranasally. The vaccine formulation may be applied to the lymphatic tissue of the nose in any convenient manner. However, it is preferred to apply it as a liquid stream or liquid droplets to the walls of the nasal passage. The intranasal composition can be formulated, for example, in liquid form as nose drops, spray, or suitable for inhalation, as powder, as cream, or as emulsion.

In another embodiment of the invention, administration is oral and the vaccine may be presented, for example, in the form of a tablet or encased in a gelatin capsule or a microcapsule.

The formulation of these modalities is general knowledge to those with skill in the art.

Liposomes provide another delivery system for antigen delivery and presentation. Liposomes are bilayered vesicles composed of phospholipids and other sterols surrounding a typically aqueous center where antigens or other products can be encapsulated. The liposome structure is highly versatile with many types range in nanometer to micrometer sizes, from about 25 nm to about 500 μm. Liposomes have been found to be effective in delivering therapeutic agents to dermal and mucosal surfaces. Liposomes can be further modified for targeted delivery by for example, incorporating specific antibodies into the surface membrane, or altered to encapsulate bacteria, viruses or parasites. The average survival time or half life of the intact liposome structure can be extended with the inclusion of certain polymers, for example polyethylene glycol, allowing for prolonged release in vivo. Liposomes may be unilamellar or multilamellar.

The vaccine composition may be formulated by: encapsulating an antigen or an antigen/adjuvant complex in liposomes to form liposome-encapsulated antigen and mixing the liposome-encapsulated antigen with a carrier comprising a continuous phase of a hydrophobic substance. If an antigen/adjuvant complex is not used in the first step, a suitable adjuvant may be added to the liposome-encapsulated antigen, to the mixture of liposome-encapsulated antigen and carrier, or to the carrier before the carrier is mixed with the liposome-encapsulated antigen. The order of the process may depend on the type of adjuvant used. Typically, when an adjuvant like alum is used, the adjuvant and the antigen are mixed first to form an antigen/adjuvant complex followed by encapsulation of the antigen/adjuvant complex with liposomes. The resulting liposome-encapsulated antigen is then mixed with the carrier. The term “liposome-encapsulated antigen” may refer to encapsulation of the antigen alone or to the encapsulation of the antigen/adjuvant complex depending on the context. This promotes intimate contact between the adjuvant and the antigen and may, at least in part, account for the immune response when alum is used as the adjuvant. When another is used, the antigen may be first encapsulated in liposomes and the resulting liposome-encapsulated antigen is then mixed into the adjuvant in a hydrophobic substance.

In formulating a vaccine composition that is substantially free of water, antigen or antigen/adjuvant complex is encapsulated with liposomes and mixed with a hydrophobic substance. In formulating a vaccine in an emulsion of water-in-a hydrophobic substance, the antigen or antigen/adjuvant complex is encapsulated with liposomes in an aqueous medium followed by the mixing of the aqueous medium with a hydrophobic substance. In the case of the emulsion, to maintain the hydrophobic substance in the continuous phase, the aqueous medium containing the liposomes may be added in aliquots with mixing to the hydrophobic substance.

In all methods of formulation, the liposome-encapsulated antigen may be freeze-dried before being mixed with the hydrophobic substance or with the aqueous medium as the case may be. In some instances, an antigen/adjuvant complex may be encapsulated by liposomes followed by freeze-drying. In other instances, the antigen may be encapsulated by liposomes followed by the addition of adjuvant then freeze-drying to form a freeze-dried liposome-encapsulated antigen with external adjuvant. In yet another instance, the antigen may be encapsulated by liposomes followed by freeze-drying before the addition of adjuvant. Freeze-drying may promote better interaction between the adjuvant and the antigen resulting in a more efficacious vaccine.

Formulation of the liposome-encapsulated antigen into a hydrophobic substance may also involve the use of an emulsifier to promote more even distribution of the liposomes in the hydrophobic substance. Typical emulsifiers are well-known in the art and include mannide oleate (Arlacel™ A), lecithin, Tween™ 80, Spans™ 20, 80, 83 and 85. The emulsifier is used in an amount effective to promote even distribution of the liposomes. Typically, the volume ratio (v/v) of hydrophobic substance to emulsifier is in the range of about 5:1 to about 15:1.

Microparticles and nanoparticles employ small biodegradable spheres which act as depots for vaccine delivery. The major advantage that polymer microspheres possess over other depot-effecting adjuvants is that they are extremely safe and have been approved by the Food and Drug Administration in the US for use in human medicine as suitable sutures and for use as a biodegradable drug delivery system (Langer R. Science. 1990; 249(4976): 1527-33). The rates of copolymer hydrolysis are very well characterized, which in turn allows for the manufacture of microparticles with sustained antigen release over prolonged periods of time (O'Hagen, et al., Vaccine, 1993; 11:965-9).

Parenteral administration of microparticles elicits long-lasting immunity, especially if they incorporate prolonged release characteristics. The rate of release can be modulated by the mixture of polymers and their relative molecular weights, which will hydrolyze over varying periods of time. Without wishing to be bound to theory, the formulation of different sized particles (1 μm to 200 μm) may also contribute to long-lasting immunological responses since large particles must be broken down into smaller particles before being available for macrophage uptake. In this manner a single-injection vaccine could be developed by integrating various particle sizes, thereby prolonging antigen presentation and greatly benefiting livestock producers.

In some applications an adjuvant or excipient may be included in the vaccine formulation. Montanide™ (Incomplete Freund's adjuvant) and alum for example, are preferred adjuvants for human use. The choice of the adjuvant will be determined in part by the mode of administration of the vaccine. A preferred mode of administration is intramuscular administration. Another preferred mode of administration is intranasal administration. Non-limiting examples of intranasal adjuvants include chitosan powder, PLA and PLG microspheres, QS-21, AS02A ,calcium phosphate nanoparticles (CAP); mCTA/LTB (mutant cholera toxin E112K with pentameric B subunit of heat labile enterotoxin), and detoxified E. Coli derived labile toxin.

The adjuvant used may also be, theoretically, any of the adjuvants known for peptide- or protein-based vaccines. For example: inorganic adjuvants in gel form (aluminium hydroxide/aluminium phosphate, Warren, H. S., et al., 1986, Ann Rev Immunol 4, 369-388; calcium phosphate, Relyvelt, E. H., 1986, Develop Biol Standard, 65, 131-136); bacterial adjuvants such as monophosphoryl lipid A (Ribi, E., 1984, J Biol Res Mod, 3, 1-9; Baker, P. J., et al., 1988, Infect Immun 56, 3064-3066) and muramyl peptides (Ellouz, F., et al., 1974, Biochem Biophys Res Commun 59, 1317-1325; Allison, A. C., and Byars, N. E., 1991, Mol Immunol 28, 279-284; Waters, R. V., et al., 1986, Infect Immun 52, 816-825); particulate adjuvants such as the so-called ISCOMS (“immunostimulatory complexes”, Mowat, A. M., and Donachie, A. M., 1991, Immunol Today 12, 383-385; Takahashi, H., et al., 1990, Nature 344, 873-875; Thapar, M. A., et al., 1991, Vaccine 9, 129-133), liposomes (Mbawuike, I. N., et al., 1990, Vaccine 8, 347-352; Abraham, E., 1992, Vaccine 10, 461-468; Phillips, N. C. and Emili, A, 1992, Vaccine 10, 151-158; Gregoriadis, G., 1990, Immunol Today 11, 89-97) and biodegradable microspheres (Marx, P. A., et al., 1993, Science 28, 1323-1327); adjuvants based on oil emulsions and emulsifiers such as IFA (“Incomplete Freund's adjuvant” (Stuart-Harris, C. H., 1969, Bull WHO 41, 617-621; Warren, H. S., et al., 1986, Ann Rev Immunol 4, 369-388), SAF (Allison, A. C., and Byars, N. E., 1991, Mol Immunol 28, 279-284), saponines (such as QS-21; Newman, M. J., et al., 1992, J Immunol 148, 23572362), squalene/squalane (Allison, A. C., and Byars, N. E., 1991, Mol Immunol 28, 279-284); synthetic adjuvants such as non-ionic block copolymers (Hunter, R., et al., 1991, Vaccine 9, 250-255), muramyl peptide analogs (Azuma, I., 1992, Vaccine 10, 1000-1004), synthetic lipid A (Warren, H. S., et al., 1986, Ann Rev Immunol 4, 369-388; Azuma, I., 1992, Vaccine 10, 1000-1004), synthetic polynucleotides (Harrington, D. G., et al., 1978, Infect Immun 24, 160-166) and polycationic adjuvants (WO 97/30721).

Adjuvants for use with immunogens of the present invention include aluminum or calcium salts (for example hydroxide or phosphate salts). A particularly preferred adjuvant for use herein is an aluminum hydroxide gel such as Alhydrogel™. Calcium phosphate nanoparticles (CAP) is an adjuvant being developed by Biosante, Inc (Lincolnshire, Ill.). The immunogen of interest can be either coated to the outside of particles, or encapsulated inside on the inside (He et al., 2000, Clin. Diagn. Lab. Immunol., 7,899-903).

Another adjuvant for use with an immunogen of the present invention is an emulsion. A contemplated emulsion can be an oil-in-water emulsion or a water-in-oil emulsion. In addition to the immunogenic chimer protein particles, such emulsions comprise an oil phase of squalene, squalane, peanut oil or the like as are well known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate.

Such emulsions are for example water-in-oil emulsions that comprise squalene, glycerol and a surfactant such as mannide mono-oleate (Arlacel™ A), optionally with squalane, emulsified with the chimer protein particles in an aqueous phase. Alternative components of the oil-phase include alpha-tocopherol, mixed-chain di- and tri-glycerides, and sorbitan esters. Well-known examples of such emulsions include Montanide™ ISA-720, and Montanide™ ISA 703 (Seppic, Castres, France. Other oil-in-water emulsion adjuvants include those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. Nos. 4,539,205, 4,643,992, 5,011,828 and 5,093,318. 7-allyl-8-oxoguanosine (loxoribine) has been shown to be particularly effective in inducing an antigen-(immunogen-) specific response.

A useful adjuvant includes monophosphoryl lipid A (MPL®), 3-deacyl monophosphoryl lipid A (3D-MPL®), a well-known adjuvant manufactured by Corixa Corp. of Seattle, formerly Ribi Immunochem, Hamilton, Mont. The adjuvant contains three components extracted from bacteria: monophosphoryl lipid (MPL) A, trehalose dimycolate (TDM) and cell wall skeleton (CWS) (MPL+TDM+CWS) in a 2% squalene/Tween™ 80 emulsion. This adjuvant can be prepared by the methods taught in GB 2122204B.

Other compounds are structurally related to MPL® adjuvant called aminoalkyl glucosamide phosphates (AGPs) such as those available from Corixa Corp under the designation RC-529™ adjuvant {2-[(R)-3-tetra-decanoyloxytetradecanoylamino]-ethyl-2-deoxy-4-O-phosphon-o-3-O-[(R)-3-tetra-decanoyloxytetra-decanoyl]-2-[(R)-3-tetra-decanoyloxytet-radecanoyl-amino]-p-D-glucopyranoside triethylammonium salt}. An RC-529 adjuvant is available in a squalene emulsion sold as RC-529SE and in an aqueous formulation as RC-529AF available from Corixa Corp. (see U.S. Pat. Nos. 6,355,257, 6,303,347 and 6,113,918; and U.S. Patent Publication No. 2003-0092643).

Further contemplated adjuvants include synthetic oligonucleotide adjuvants containing the CpG nucleotide motif one or more times (plus flanking sequences) available from Coley Pharmaceutical Group. The adjuvant designated QS21, available from Aquila Biopharmaceuticals, Inc., is an immunologically active saponin fractions having adjuvant activity derived from the bark of the South American tree Quillaja Saponaria Molina (e.g. Quil™ A), and the method of its production is disclosed in U.S. Pat. No. 5,057,540. Derivatives of Quil™ A, for example QS21 (an HPLC purified fraction derivative of Quil™ A also known as QA21), and other fractions such as QA17 are also disclosed. Semi-synthetic and synthetic derivatives of Quillaja Saponaria Molina saponins are also useful, such as those described in U.S. Pat. No. 5,977,081 and U.S. Pat. No. 6,080,725. The adjuvant denominated MF59 available from Chiron Corp. is described in U.S. Pat. No. 5,709,879 and U.S. Pat. No. 6,086,901.

Muramyl dipeptide adjuvants are also contemplated and include N-acetyl-muramyl-L-threonyl-D-isoglutamine (thur-MDP), N-acetyl-nor-muramyl-L-alanyl-D-isoglutamine (CGP 11637, referred to as nor-MDP), and N-acetylmuramyl-L-alanyl-D-isoglutaminyl-L-alanine-2-(1′-2′-dipalmityol-s-n-glycero-3-hydroxyphosphoryloxy) ethylamine ((CGP) 1983A, referred to as MTP-PE). The so-called muramyl dipeptide analogues are described in U.S. Pat. No. 4,767,842.

Other adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). Adjuvant SBAS2 (now ASO2) available from SKB (now Glaxo-SmithKline) contains QS21 and MPL in an oil-in-water emulsion is also useful. Alternative adjuvants include those described in WO 99/52549 and non-particulate suspensions of polyoxyethylene ether (UK Patent Application No. 9807805.8).

The use of an adjuvant that contains one or more agonists for toll-like receptor-4 (TLR-4) such as an MPL® adjuvant or a structurally related compound such as an RC-529® adjuvant or a Lipid A mimetic, alone or along with an agonist for TLR-9 such as a non-methylated oligo deoxynucleotide-containing the CpG motif is also optional.

Another type of adjuvant mixture comprises a stable water-in-oil emulsion further containing aminoalkyl glucosamine phosphates such as described in U.S. Pat. No. 6,113,918. Of the aminoalkyl glucosamine phosphates the molecule known as RC-529 {2-[(R)-3-tetradecanoyloxytetradecanoylamino]ethyl 2-deoxy-4-O-phosphono-3-O-[(R)-3-tetradecanoyloxy-tetradecanoyl]-2-[(R)-3-tetradecanoyloxytetra-decanoylamino]-p-D-glucopyranoside triethylammonium salt.)} is preferred. One particular water-in-oil emulsion is described in WO 99/56776.

Adjuvants are utilized in an adjuvant amount, which can vary with the adjuvant, host animal and immunogen. Typical amounts can vary from about 1 μg to about 1 mg per immunization. Those skilled in the art know that appropriate concentrations or amounts can be readily determined.

Vaccine compositions comprising an adjuvant based on oil in water emulsion is also included within the scope of the present invention. The water in oil emulsion may comprise a metabolisable oil and a saponin, such as for example as described in U.S. Pat. No. 7,323,182.

According to several embodiments, the vaccine compositions according to the present invention may contain one or more adjuvants, characterized in that it is present as a solution or emulsion which is substantially free from inorganic salt ions, wherein said solution or emulsion contains one or more water soluble or water-emulsifiable substances which is capable of making the vaccine isotonic or hypotonic. The water soluble or water-emulsifiable substances may be, for example, selected from the group consisting of: maltose; fructose; galactose; saccharose; sugar alcohol; lipid; and combinations thereof.

The compositions of the present invention comprise according to several specific embodiments a proteosome adjuvant. The proteosome adjuvant comprises a purified preparation of outer membrane proteins of meningococci and similar preparations from other bacteria. These proteins are highly hydrophobic, reflecting their role as transmembrane proteins and porins. Due to their hydrophobic protein-protein interactions, when appropriately isolated, the proteins form multi-molecular structures consisting of about 60-100 nm diameter whole or fragmented membrane vesicles. This liposome-like physical state allows the proteosome adjuvant to act as a protein carrier and also to act as an adjuvant.

The use of proteosome adjuvant has been described in the prior art and is reviewed by Lowell G H in “New Generation Vaccines”, Second Edition, Marcel Dekker Inc, New York, Basel, Hong Kong (1997) pages 193-206. Proteosome adjuvant vesicles are described as comparable in size to certain viruses which are hydrophobic and safe for human use. The review describes formulation of compositions comprising non-covalent complexes between various antigens and proteosome adjuvant vesicles which are formed when solubilizing detergent is selectably removed using exhaustive dialysis technology.

The present invention also encompasses within its scope the preparation and use of DNA vaccines. Vaccination methods and compositions of this type are well known in the art and are comprehensively described in many different articles, monographs and books (see, for example, chapter 11 of “Molecular Biotechnology: principles and applications of recombinant DNA” eds. B. R. Glick & J. J. Pasternak, ASM Press, Washington, D.C., 2^{nd} edition, 1998). In principle, DNA vaccination is achieved by cloning the cDNAs for the desired immunogen into a suitable DNA vaccine vector, such as the pVAC vector (Invivogen), using codons optimized for expression in human. In the case of pVAC, the desired immunogenic proteins are targeted and anchored to the cell surface by cloning the gene of interest in frame between the IL2 signal sequence and the C-terminal transmembrane anchoring domain of human placental alkaline phosphatase. The use of other immune enhancers, including adjuvants or cloning in frame other immune enhancing cytokines, together with the DNA vaccines is also within the scope of the present invention. Such DNA vaccine vectors are specifically designed to stimulate humoral immune responses by intramuscular injection. The antigenic peptide produced on the surface of muscle cells is taken up by antigen presenting cells (APCs), processed and presented to the immune system T helper cells through the major histocompatibility complex (MHC) class II molecules.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

The aforementioned adjuvants are substances that can be used to augment a specific immune response. Normally, the adjuvant and the composition are mixed prior to presentation to the immune system, or presented separately, but into the same site of the subject being vaccinated. Adjuvants that may be usefully employed in the preparation of vaccines include: oil adjuvants (for example, Freund's complete and incomplete adjuvants, that will be used in animal experiments only and is forbidden from use in humans), mineral salts, alum, silica, kaolin, and carbon, polynucleotides and certain natural substances of microbial origin. An additional mode of antigen delivery may include an encapsulation technique, which involves complex coacervation of gelatin and chondroitin sulfate (Azhari R, Leong K W. 1991. Complex coacervation of chondroitin sulfate and gelatin and its use for encapsulation and slow release of a model protein. Proc. Symp. Control. Rel. 18: 617; Brown K E, Leong K, Huang C H, Dalal R, Green G D, Haimes H B, Jimenez P A, Bathon J. 1998. Gelatin/chondroitin 6-sulfate microspheres for delivery of therapeutic proteins to thejoint. Arthritis Rheum 41: 2185-2195).

Further examples of materials and methods useful in the preparation of vaccine compositions are well known to those skilled in the art. In addition, further details may be gleaned from Remington's Pharmaceutical Sciences, Mack Publishing Co, Easton, Pa., USA, 20^{th} edition 2000.

The S. pneumoniae cell-wall and/or cell-membrane proteins for use in working the present invention may be obtained by directly purifying said proteins from cultures of S. pneumoniae by any of the standard techniques used to prepare and purify cell-surface proteins. Suitable methods are described in many biochemistry text-books, review articles and laboratory guides, including inter alia “Protein Structure: a practical approach” ed. T. E. Creighton, IRL Press, Oxford, UK (1989).

However, it is to be noted that such an approach suffers many practical limitations that present obstacles for producing commercially-viable quantities of the desired proteins. The S. pneumoniae proteins of the present invention may therefore be more conveniently prepared by means of recombinant biotechnological means, whereby the gene for the S. pneumoniae protein of interest is isolated and inserted into an appropriate expression vector system (such as a plasmid or phage), which is then introduced into a host cell that will permit large-scale production of said protein by means of, for example, overexpression.

As a first stage, the location of the genes of interest within the S. pneumoniae genome may be determined by reference to a complete-genome database such as the TIGR database that is maintained by the Institute for Genomic Research. The selected sequence may, where appropriate, be isolated directly by the use of appropriate restriction endonucleases, or more effectively by means of PCR amplification. Suitable techniques are described in, for example, U.S. Pat. Nos. 4,683,195, 4,683,202, 4,800,159 and 4,965,188, as well as in Innis et al. eds., PCR Protocols: A guide to method and applications. Alternatively, the gene may be chemically synthesized with codons optimized to the expression system actually used (i.e. E. coli). For DNA vaccines, codons are optimized for expression in human.

Following amplification and/or restriction endonuclease digestion, the desired gene or gene fragment is ligated either initially into a cloning vector, or directly into an expression vector that is appropriate for the chosen host cell type. In the case of the S. pneumoniae proteins, Escherichia coli is the most useful expression host. However, many other cell types may be also be usefully employed including other bacteria, yeast cells, insect cells and mammalian cell systems known in the art.

High-level expression of the desired protein (as intact protein sequence, modified protein sequence, fragment of thereof), within the host cell may be achieved in several different ways (depending on the chosen expression vector) including expression as a fusion protein (e.g. with factor Xa or thrombin), expression as a His-tagged protein, dual vector systems, expression systems leading to incorporation of the recombinant protein inside inclusion bodies etc. The recombinant protein will then need to be isolated and purified from the cell membrane, interior cellular soluble fraction, inclusion body or (in the case of secreted proteins) the culture medium, by one of the many methods known in the art.

All of the above recombinant DNA and protein purification techniques are well known to all skilled artisans in the field, the details of said techniques being described in many standard works including “Molecular cloning: a laboratory manual” by Sambrook, J., Fritsch, E. F. & Maniatis, T., Cold Spring Harbor, N.Y., 2^{nd} ed., 1989, which is incorporated herein by reference in its entirety.

As disclosed and explained hereinabove, each of the abovementioned embodiments of the invention may be based on the use of one or more intact, full length, cell wall and/or cell membrane proteins or, in the alternative, or in addition thereto, fragments, derivatives and modifications of said full length proteins. Fragments may be obtained by means of recombinant expression of selected regions of the cell wall protein gene(s). Derivatives of the full length proteins or fragments thereof may be obtained by introducing non-native sequences within the DNA sequences encoding said proteins, followed by expression of said derivatized sequences. Derivatives may also be produced by conjugating non-native groups to the amino residue side chains of the cell wall proteins or protein fragments, using standard protein modification techniques. Modified cell wall proteins and protein fragments for use in the present invention may also be obtained by the use of site-directed mutagenesis techniques. Such techniques are well known in the art and are described, for example, in “Molecular cloning: a laboratory manual” by Sambrook, J., Fritsch, E. F. & Maniatis, T., Cold Spring Harbor, N.Y., 2^{nd} ed., 1989. Of particular interest is the use of one or more of the preceding techniques to create fragments or derivatives possessing the desired epitopic sites, but lacking other domains which are responsible for adverse effects such as suppression of cellular immune responses. It is to be emphasized that all of the immediately preceding discussion of fragments, derivatives and mutants of the cell wall proteins disclosed herein are to be considered as an integral part of the present invention.

S. pneumoniae infections are common in children under the five years of age mainly under two years of age. The infants' antibody production is known to be produced at 6 months of age. The present invention is based in part on a study performed with sera obtained longitudinally from children at 18, 30 and 42 months of age, attending day care centers, which are exposed to the bacteria. The children's sera were screened for change, with age, of the presence or amount of antibodies to specific cell wall/membrane proteins. Antibodies to specific proteins which were absent or low in sera of younger children and appear or increase with age identified proteins that now would be considered as candidate for vaccine development for protecting infants against S. pneumoniae. Without wishing to be bound to any theory it is suspected that the immune response of younger children to the proteins in the context of the bacterium is also not efficient. Since the increase in the response to these proteins is in reciprocal correlation with disease it was assumed that immunization with these proteins will elicit a protective immune response. Each of the proteins in the set disclosed for the first time in the present application as being associated with age-dependent immune response to the bacteria may elicit protective immune response against the bacteria at all ages to all subjects, including infants, elderly and immunocompromised subjects.

The following examples are provided for illustrative purposes and in order to more particularly explain and describe the present invention. The present invention, however, is not limited to the particular embodiments disclosed in the examples.

### EXAMPLE 1

Prevention of S. pneumoniae Infection in Mice by Inoculation with S. pneumoniae Cell Wall Protein Fractions

### Methods:

Bacterial Cells: The bacterial strain used in this study was an S. pneumoniae serotype 3 strain and R6. The bacteria were plated onto tryptic soy agar supplemented with 5% sheep erythrocytes and incubated for 17-18 hours at 37° C. under anaerobic conditions. The bacterial cells were then transferred to Todd-Hewitt broth supplemented with 0-5% yeast extract and grown to mid-late log phase. Bacteria were harvested and the pellets were stored at −70° C.
Purification of Cell Wall Proteins: Bacterial pellets were resuspended in phosphate buffered saline (PBS). The resulting pellets were then treated with mutanolysin to release cell wall components. Supernatants containing the CW proteins were then harvested. Subsequently, the bacteria were sonicated, centrifuged and the resulting pellet containing the bacteria membranes (m) were lysed with 0.5% triton X-100.
Fractionation of the Cell Wall Protein Mixture: Cell wall protein-containing supernatants were allowed to adhere to fetuin (a highly glycosylated pan-lectin binding protein) that was covalently bound to a sepharose column. Non-adherent molecules, obtained from the flow-through fraction were predominantly non-lectin molecules, while the column-adherent lectins were eluted with 50 mM ammonium acetate at pH 3.5.
Experimental: S. pneumoniae cell wall (CW) proteins were separated into lectin (CW-L) and non-lectin (NL) fractions by fetuin affinity chromatography, as described hereinabove. C57BL/6 and BALB/c mice were vaccinated with S. pneumoniae total CW (CW-T), CW-L and CW-NL protein preparations mixed with Freund's adjuvant, by means of the following procedure: each mouse was primed with 25 micrograms of CW-T, CW-NL and CW-L protein preparations intramuscularly, with complete Freund's adjuvant (CFA) and boosted with incomplete Freund's adjuvant (IFA), 4 and 7 weeks following priming. Western blots of the abovementioned protein preparations were probed with sera obtained a week after the last immunization. Animals were then challenged intranasally (IN) or intraperitoneally (IP) with 10⁸ cfu of S. pneumoniae serotype 3, that caused 100% mortality in control mice immunized with CFA and boosted with IFA only within 96 hours post-inoculation. Vaccination with CW-L elicited partial protection against S. pneumoniae IN and IP challenge (50% and 45% respectively). Vaccination with CW-T and CW-NL proteins elicited 70% and 65% protection against IP challenge, respectively. Vaccination with CW-T and CW-NL proteins elicited 85% and 50% protection against IN inoculation, respectively.

### EXAMPLE 2

Determination of Age-Related Immunoreactivity to S. pneumoniae Surface Proteins

The following study was carried out in order to investigate the age-related development of immunoreactivity to S. pneumoniae cell wall and cell membrane proteins.

Operating as described hereinabove in Example 1, a fraction containing cell wall proteins was obtained from a clinical isolate of S. pneumoniae. In addition, cell membrane proteins were recovered by solubilizing the membrane pellet in 0.5% Triton X-100. The cell wall and cell membrane proteins were separated by means of two-dimensional gel electrophoresis, wherein the proteins were separated using polyacrylamide gel isoelectric focusing in one dimension, and sodium dodecyl sulfate polyacrylamide gel electrophoresis (SDS-PAGE) in the other dimension. The separated proteins were either transferred to a nitrocellulose membrane or directly stained with Coomassie Brriliant blue.

Sera were collected longitudinally from healthy children attending day-care centers at 18, 30 and 42 months of age. Starting at 12 months of age, nasopharyngeal swabs were taken from the children on a bimonthly schedule over the 2-5 years of the study. Pneumococcal isolates were characterized by inhibition with optochin and a positive slide agglutination test (Phadebact, Pharmacia Diagnostics). In addition, sera were collected from healthy adults.

The ability of serum prepared from the above-mentioned blood samples to recognize the separated S. pneumoniae proteins was investigated by Western blot analysis according to the methods described by Rapola S. et al. (J. Infect. Dis., 2000, 182: 1146-52). Putative identification of the separated protein spots obtained following the 2D-electrophoresis was achieved by the use of the Matrix Assisted Laser Desorption/Ionization mass spectrometery (MALDI-MS). The results of the above analysis are summarized in the following table:

TABLE 1
Age-dependent immunoreactivity to S. pneumoniae surface proteins

| Spot | Proteins/ | | Age (years) | | | |
|---|---|---|---|---|---|---|
| no. | spot | Homology to | 1.5 | 2.5 | 3.5 | adult |
| 1 | 2 | DNA K/phosphoenolpyruvate protein | * | * | * | * |
| | | Phosphoesterase | | | | |
| 3 | 1 | Trigger factor | * | * | * | * |
| 4 | 2 | 60 KDa chaperonin (GroEl protein) | ** | * | ** | *** |
| | | Eleongation factor G/tetracycline resistance | | | | |
| | | protein teto (TET(O)) | | | | |
| 7 | 2 | Glutamyl-tRNA amidotransferase subunit | * | ** | | *** |
| | | A/N utilization sybstance protein protein A | | | | |
| 11 | 2 | Oligopeptide-binding protein amiA/aliA/aliB | | | | |
| | | precursor | | | | |
| | | Hypothetical zinc metalloproteinase in SCAA | | | | |
| | | 5′region (ORF 6) | | | | |
| 12 | 1 | Pneumolysin (thiol-activated cytolysin | * | | * | ** |
| 13 | 1 | L-lactate dehydrogenase | * | ** | * | |
| 14 | 1 | Glyceraldehyde 3-phosphate dehydrogenase | * | ** | *** | *** |
| | | (GAPDH) | | | | |
| 15 | 1 | Fructose-bisphosphate aldolase | ** | *** | *** | *** |
| 16 | 1 | UDP-glucose 4-epimerase | ** | * | | |
| 17 | 2 | Elongation factor G/tetracycline resistance | * | ** | | |
| | | protein teto (TET(O)) | | | | |
| 18 | 1 | Pyruvat oxidase | *** | *** | *** | *** |
| 22 | 1 | Glutamyl-tRNA synthetase | | * | ** | |
| 23 | 1 | NADP-specific glutamate dehydrogenase | * | | * | * |
| 24 | 1 | Glyceraldehydes 3-phosphate dehydrogenase | * | ** | *** | **** |
| | | (GAPDH) | | | | |
| 25 | 1 | Enolase (2-phosphoglycerate dehydratase) | * | ** | ** | ** |
| 27 | 1 | Phosphoglycerate kinase | * | ** | ** | ** |
| 29 | 1 | Glucose-6-phosphate isomerase | | * | * | ** |
| 30 | 2 | 40S ribosomal protein | | | | |
| | | S1/6-phosphogluconate dehydrogenase | | | | |
| 31 | 1 | Aminopeptidase C | | | | |
| 33 | | Carbamoyl-phosphate synthase | | * | ** | *** |
| 57/65 | | Aspartate carbamoyltransferase | * | * | ** | ** |
| 58 | | 30S ribosomal protein S2 | ** | * | | |

* Low;
** intermediate;
*** high

The data presented in the preceding table indicate that there is an age-dependent development of immunoreactivity to several S. pneumoniae cell wall and cell surface proteins.

Ling et al. (Clin. Exp. Immunol. 138:290-298, 2004) further describes identification of S. pneumoniae vaccine candidates. As shown in table 2, it was found that the antigenic proteins from the enriched cell wall extract fell into three groups. The first group comprised proteins with low immunogenicity. The second group consists of antigens for which the immunogenicity seemed to increase with age of children attending day-care centers, while the third group of proteins was highly antigenic with all sera tested. The existence of serum antibodies to a certain bacterial protein does not necessarily indicate their capacity to elicit protective immune response against the bacteria. However, the increase in the antibody response to bacterial proteins which coincides with the diminution in morbidity described in children encouraged to test these antigens for their ability to elicit protection against S. pneumoniae. It is concluded that the immunogenic enzymes with an age dependent increase in antigenicity of S. pneumoniae found in enriched cell wall and membrane extract may represent a novel class of vaccine candidates. As shown herein for the first time many of these identified proteins/enzymes elicit protective level immune responses in mice and afford significant protection against respiratory challenge with virulent S. pneumoniae.

TABLE 2
Identification of S. pneumoniae surface proteins with age-dependent immunogenicity

| | | | | | | Immunoreactivity | | | |
|---|---|---|---|---|---|---|---|---|---|
| | MALDI-TOF analysis | | | | | Age (months) | | | |
| Spot | Homology | Acc. number | Mascot | MW | pI | 1.5 | 2.5 | 3.5 | Adult |
| Proteins with low immunogenicity | | | | | | | | | |
| 1 | D NAK | NP_345035 | 173 | 64.8 | 4.6 | * | * | | |
| 23 | NADP-specific | NP_345769 | 186 | 49 | 5.3 | * | * | | * |
| | glutamate dehydrogenase | | | | | | | | |
| Proteins with increased immunogenicity | | | | | | | | | |
| 7 | Glutamyl-tRNA | NP_344959 | 83 | 52 | 4.9 | * | ** | ** | *** |
| | Amidotransferase | | | | | | | | |
| | subunit A | | | | | | | | |
| 13 | L-lactate dehydrogenase | NP_345686 | 134 | 35.9 | 5.2 | * | ** | * | ** |
| 14 | Glyceraldehyde 3- | NP_346439 | 350 | 37.1 | 5.7 | * | ** | *** | *** |
| | phosphate | | | | | | | | |
| | dehydrogenase | | | | | | | | |
| 15 | Fructose-bisphosphate | NP_345117 | 106 | 31.5 | 5 | ** | *** | *** | *** |
| | aldolase | | | | | | | | |
| 16 | UDP-glucose 4- | NP_346051 | 116 | 37.5 | 4.8 | ** | * | * | ** |
| | epimerase | | | | | | | | |
| 22 | Glutamyl-tRNA | NP_346492 | 194 | 56 | 4.9 | | * | ** | ** |
| | synthetase | | | | | | | | |
| 27 | Phosphoglycerate kinase | NP_345017 | 109 | 41.9 | 4.9 | * | ** | ** | ** |
| 29 | Glucose-6-phosphate | NP_346493 | 96 | 51.3 | 5.2 | | * | * | ** |
| | isomerase | | | | | | | | |
| 30 | 6-phosphogluconate | NP_344902 | 58 | 53.7 | 4.9 | | | ** | ** |
| | dehydrogenase | | | | | | | | |
| 31 | Aminopeptidase C | NP_344819 | 120 | 33.7 | 4.8 | | | ** | ** |
| x | Hypothetical protein | NP_358083 | | 15 | 5.2 | | | * | ** |
| 33 | Carbamoyl-phosphate | NP_345739 | 230 | 116.5 | 4.8 | | * | ** | *** |
| | synthase | | | | | | | | |
| 65 | Aspartate | NP_345741 | 44 | 34.7 | 5.1 | * | * | ** | ** |
| | carbamoyltransferase | | | | | | | | |
| Proteins with high immunogenicity | | | | | | | | | |
| 18 | Pyruvate oxidase | NP_345231 | 168 | 65.3 | 5.1 | *** | *** | *** | *** |
| 25 | Enolase (2- | NP_345598 | 215 | 47.1 | 4.7 | ** | ** | ** | ** |
| | phosphoglycerate | | | | | | | | |
| | dehydratase) | | | | | | | | |

* Low;
** intermediate;
*** high

The extent of surface protein recognition by the sera was determined by the optical density as measured by the imager used in our study (αInnotech).

### EXAMPLE 3

Prevention of S. pneumoniae Infection in Mice with Recombinantly-expressed S. pneumoniae Cell Surface Proteins

Glycolytic enzymes associated with the cell surface of Streptococcus pneumoniae are antigenic in humans and elicit protective immune responses in the mouse.

The glycolytic enzymes fructose-bisphosphate aldolase (FBA, NP_{—}345117, SEQ ID NO:13), and Glyceraldehide 3 phosphate dehydrogenase (GAPDH, NP 346439, SEQ ID NO:12), which are associated with the cell surface of S. pneumoniae, were used to immunize mice against S. pneumonia as described in Ling et al., Clin. Exp. Immunol. 138:290-298. 2004. It was shown that both proteins, which are antigenic in humans, elicit cross-strain protective immunity in mice.

Cloning of Immunogenic S. pneumoniae Surface Proteins: S. pneumoniae fructose-bisphosphate aldolase (hereinafter referred to as “aldolase”) and GAPDH proteins were cloned into the pHAT expression vector (BD Biosciences Clontech, Palo Alto, Calif., USA; HAT Vectors encode polyhistidine epitope tag in which the 6 histidine are not consecutive: Lys Asp His Leu Ile His Asn Val His Lys Glu His Ala His Ala His Asn Lys), and expressed in E. coli BL21 cells (Promega Corp., USA) using standard laboratory procedures. Following lysis of the BL21 cells, recombinant proteins were purified by the use of immobilized metal affinity chromatography (IMAC) on Ni-NTA columns (Qiagen) and eluted with imidazole. In a separate set of experiments, S. pneumoniae aldolase cDNAs were cloned into the pVAC expression vector (Invivogen), a DNA vaccine vector specifically designed to stimulate a immune response by intramuscular injection. Antigenic proteins are targeted and anchored to the cell surface by cloning the gene of interest in frame between the IL2 signal sequence and the C-terminal transmembrane anchoring domain of human placental alkaline phosphatase. The antigenic peptide produced on the surface of muscle cells is taken up by antigen presenting cells (APCs) and processed to be presented to the T helper cells by the major histocompatibility complex (MHC) class II molecules.

Immunization: BALB/c and C57BL/6 mice (7 week old females) were intraperitonealy immunized with 25 micrograms of either recombinant aldolase or recombinant GAPDH proteins together with either Freund's complete adjuvant (CFA) or an alum adjuvant. In a separate set of experiments, mice of the aforementioned strains were intramuscularly immunized with 50 micrograms of the pVAC-aldolase or pVAC-GAPDH constructs that were described hereinabove.

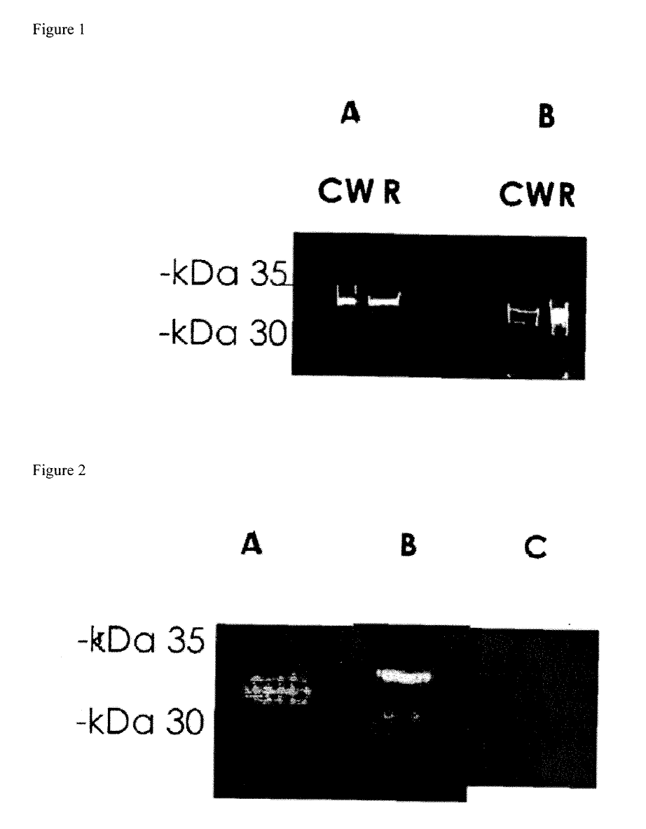

Assessment of Immunogenicity: The immunogenicity of recombinant S. pneumoniae aldolase and GAPDH proteins was assessed by Western blot assay using serum of mice that had been immunized with either total cell wall proteins (CW-T) or with one of the recombinant proteins (as described hereinabove). The results obtained (FIG. 1) indicate that the sera of the immunized animals recognized both recombinant GAPDH and aldolase proteins, and the native GAPDH and aldolase proteins present in the CW-T mixture.

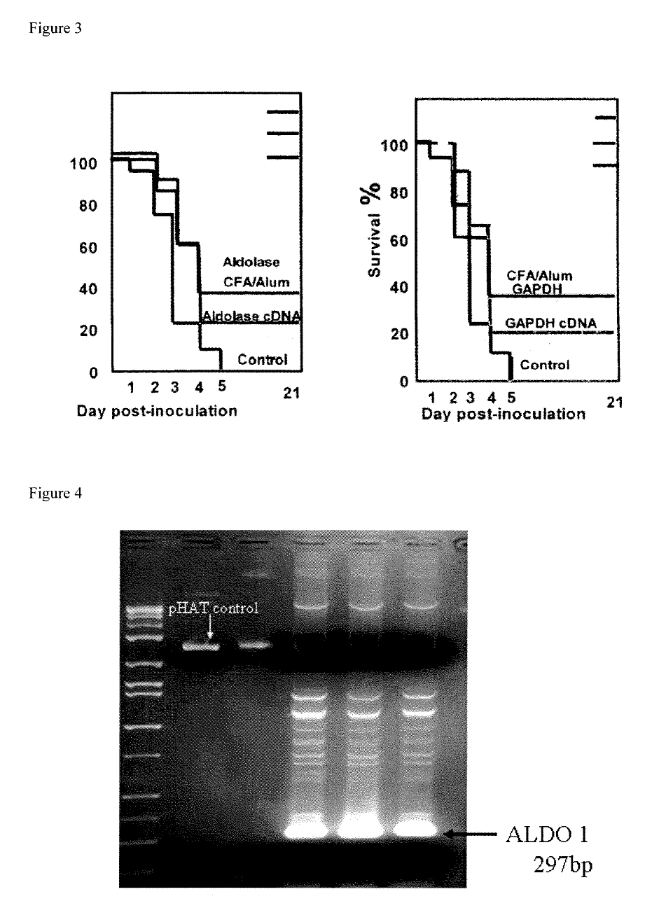

In a separate set of experiments the serum of mice that had been immunized with DNA vaccines of pVAC-aldolase or pVAC-GAPDH constructs, as described above, was used to detect native aldolase and GAPDH, respectively in Western blots obtained from SDS-PAGE separations of CW-T proteins. The results obtained (FIG. 2) indicate that inoculation with the DNA vaccines containing pVAC-based constructs is capable of eliciting an immune response. Sera of mice vaccinated with the parental pVAC plasmid (i.e. without insert) did not react with the CW-T proteins. Protective Vaccination: Following immunization with the recombinant proteins as described hereinabove, the mice were challenged intranasally with a lethal dose of 10⁸ CFU of S. pneumoniae serotype 3. Only 10% of the control animals (immunization with either CFA or alum only) survived the bacterial challenge. However, 40% of the animals immunized with the recombinant aldolase protein in CFA and 43% of the animals immunized with the same protein in alum survived the challenge. In contrast, immunization with the protein DNA K, having low immugenicity (table 2) did not elicit a protective immune response. Following immunization with the pVAC-aldolase construct, 33% of the animals survived. With regard to recombinant GAPDH, 36% of the animals immunized with this recombinant protein survived. Immunization with the pVAC-GAPDH construct, led to a survival rate of 40%, as shown in FIG. 3.

### EXAMPLE 4

S. pneumoniae Immunogenic Proteins

Operating essentially as in Example 2, the ability of serum prepared from blood samples of children aged 1.5, 2.5 and 3.5 years and adults to recognize the separated S. pneumoniae proteins was investigated by Western blot analysis according to the methods described by Rapola S. et al. (J. Infect. Dis., 2000, 182: 1146-52).

Identification of the separated protein spots obtained following the 2D-electrophoresis was achieved by the use of the Matrix Assisted Laser Desorption/Ionization mass spectrometry (MALDI-MS) technique, and comparison of the partial amino acid sequences obtained thereby with the sequences contained in the TIGR4 and/or R6 databases (maintained by The Institute for Genomic Research).

The cell surface proteins found to be immunogenic (classified according to their cellular location—cell membrane or cell wall) are summarized in the following table:

TABLE 3
list of immunogenic proteins

| Spot | | Accession | SEQ |
|---|---|---|---|
| # | Protein name | No. | ID NO |
| 1 | phosphoenolpyruvate protein phosphotransferase | NP_345645 | 4 |
| 2 | phosphoglucomutase/phosphomannomutase family | NP_346006 | 5 |
| | protein | | |
| 3 | trigger factor | NP_344923 | 6 |
| 4 | elongation factor G/tetracycline resistance protein | NP_344811 | 7 |
| | (tetO) | | |
| 6 | NADH oxidase | NP_345923 | 8 |
| 7 | Aspartyl/glutamyl-tRNA amidotransferase subunit C | NP_344960 | 9 |
| 8 | cell division protein FtsZ | NP_346105 | 10 |
| 13 | L-lactate dehydrogenase | NP_345686 | 11 |
| 14 | glyceraldehyde 3-phosphate dehydrogenase (GAPDH) | NP_346439 | 12 |
| 15 | fructose-bisphosphate aldolase | NP_345117 | 13 |
| 16 | UDP-glucose 4-epimerase | NP_346261 | 14 |
| | elongation factor Tu family protein | NP_358192 | 15 |
| 21 | Bifunctional GMP synthase/glutamine | NP_345899 | 16 |
| | amidotransferase protein | | |
| 22 | glutamyl-tRNA synthetase | NP_346492 | 17 |
| 23 | glutamate dehydrogenase | NP_345769 | 18 |
| 26 | Elongation factor TS | NP_346622 | 19 |
| 27 | phosphoglycerate kinase (TIGR4) | AAK74657 | 20 |
| 30 | 30S ribosomal protein S1 | NP_345350 | 21 |
| | 6-phosphogluconate dehydrogenase | NP_357929 | 22 |
| 31 | aminopeptidase C | NP_344819 | 23 |
| 33 | carbamoyl-phosphate synthase (large subunit) | NP_345739 | 24 |
| 57 | PTS system, mannose-specific IIAB components | NP_344822 | 25 |
| 58 | 30S ribosomal protein S2 | NP_346623 | 26 |
| 62 | dihydroorotate dehydrogenase 1B | NP_358460 | 27 |
| 65 | aspartate carbamoyltransferase catalytic subunit | NP_345741 | 28 |
| 14 | elongation factor Tu | NP_345941 | 29 |
| 19 | Pneumococcal surface immunogenic protein A (PsipA) | NP_344634 | 30 |
| 22 | phosphoglycerate kinase (R6) | NP_358035 | 31 |
| 40 | ABC transporter substrate-binding protein | NP_344690 | 32 |
| 10 | endopeptidase O | NP_346087 | 33 |
| 14 | Pneumococcal surface immunogenic protein B (PsipB) | NP_358083 | 34 |
| | Pneumococcal surface immunogenic protein C (PsipC) | NP_345081 | 35 |

### EXAMPLE 5

Preparation of an S. pneumoniae Fructose Bisphosphate Aldolase Fragment

A peptide referred to as ALDO 1, corresponding to the first 294 nucleotides of the coding sequence of the fructose bisphosphate aldolase gene (SP0605 Streptococcus pneumoniae TIGR4) (SEQ ID NO:1), was amplified from S. pneumoniae strain R6 genomic DNA by means of PCR with the following primers:

3 Forward (5′-GGT ACC ATG GCA ATC GTT TCA GCA-3′) (SEQ ID NO:2), Reverse (5′-GAG CTC ACC AAC TTC GAT ACA CTC AAG-3′) (SEQ ID NO:3).

The amplified product obtained thereby is shown in FIG. 4.

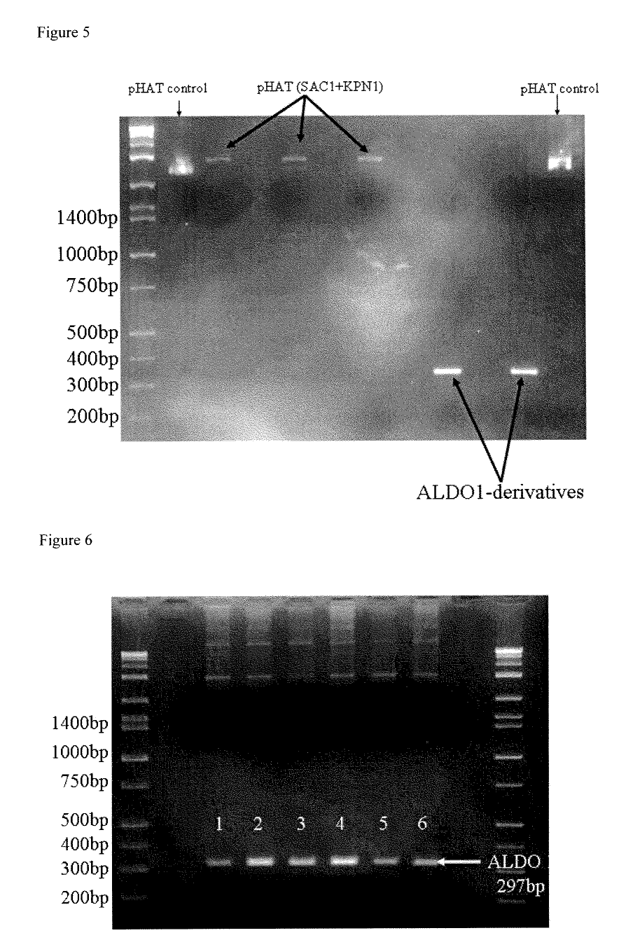

The Forward and Reverse primers, constructed according to the TIGR4 sequence contain Kpn1 and SacI recognition sequences, respectively. The primers flank the entire open reading frames. The primers were used to amplify the gene from S. pneumoniae serotype 3 strain WU2. The amplified and Kpn1-SacI (Takara Bio Inc, Shiga, Japan) digested DNA-fragments were cloned into the pHAT expression vector (BD Biosciences Clontech, Palo Alto, Calif., USA; as described in Example 3), as illustrated in FIG. 5 and transformed in DH5a UltraMAX ultracompetent E. coli cells.

Ampicillin-resistant transformants were cultured and plasmid DNA was analyzed by PCR. The pHAT-ALDO 1 vector was purified from DH5.alpha. UltraMAX cells using the Qiagen High Speed Plasmid Maxi Kit (Qiagen GMBH, Hilden, Germany) and transformed in E. coli host expression strain BL21(DE3)pLysS. PCR amplification of the ALDO 1 fragment from transformed positive colonies yielded the 297 bp fragment indicated in the gel shown in FIG. 6.

### EXAMPLE 6

Active immunization with PPP reduces nasopharyngeal and lung colonization upon intranasal challenge.

Phosphoenolpyruvate protein phosphotransferase (PPP) NP_{—}345645 (SEQ ID NO 4). The recombinant PPP protein used in this experiment was prepared as described in Example 3. The cloning of the gene was by amplification of the gene using primers constructed according to the TIGR4 sequence and the gene was amplified from S. pneumoniae serotype 3 strain WU2. The amplified gene was inserted into the pHAT vector as described in Example 3.

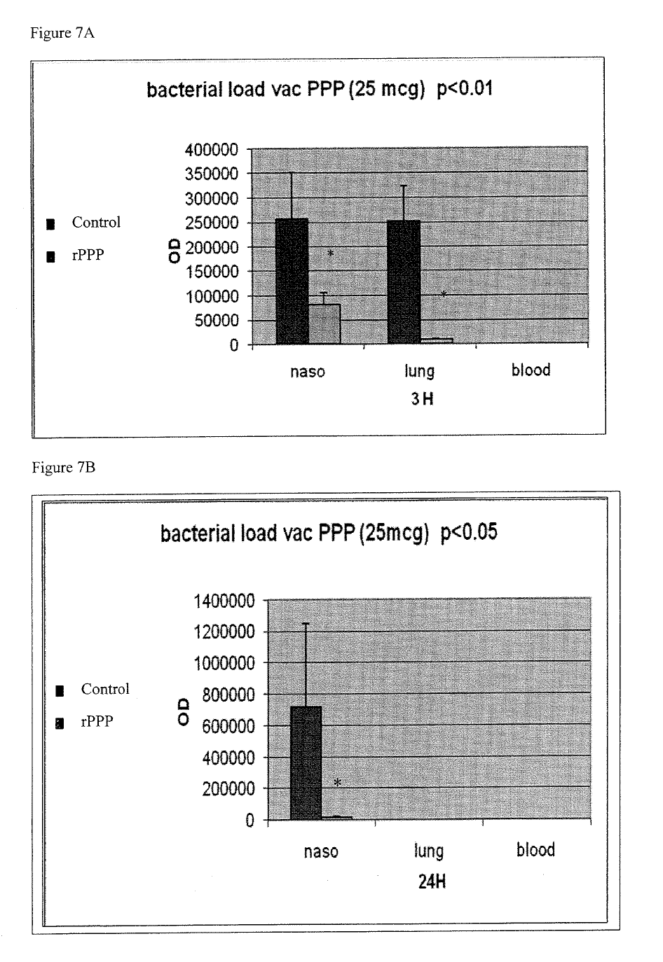

BALB/c mice (7 weeks old, 3 mice in each group) were immunized subcutaneously (SC) with PPP (25 mcg per mouse in CFA/IFA/IFA). One week after third immunization the mice were challenged intranasally (IN) with S. pneumoniae Serotype 3 strain WU2. Each mouse was anesthetized with Terrel isoflurane (MINRAD, N.Y., USA) and inoculated intra-nasally with 5×10⁷ (sub-lethal inoculum) bacteria (in 25 μl PBS). Mice were sacrificed by cervical dislocation at 3, 48 and 96 h after inoculation. The nasopharynx and the lungs were removed and homogenized in 1 ml PBS. Samples (25 μl) in serial dilutions were then plated onto blood agar plates for CFU determination. As shown in FIGS. 7A and 7B, Intranasal challenge with S. pneumoniae following active immunization with PPP reduced initial nasopharyngeal colonization and bacteria load in the lungs, measured 3 hours after bacterial challenge, significantly (p<0.01). The nasopharyngeal colonization measured 24 hours following the challenge demonstrate increased bacterial number in control (adjuvant immunized) animals but significantly reduced numbers of bacteria in the infected animals. As depicted in FIG. 7B a significantly (p<0.05) lower lung colonization was observed three hours after inoculation. All mice challenged with the sublethal doses used in this experiment were able to clear the bacteria from the lungs 24 hours following challenge.

### EXAMPLE 7

Active Immunization with Glutamyl tRNA Synthetase

Active immunization with Glutamyl tRNA synthetase (GtS, NP_{—}346492, SEQ ID NO:17) using alum as adjuvant is described in Mizrachi et al., J Infect Dis. 196,945-53, 2007. The cloning of the gene was by amplification of the gene using primers constructed according to the TIGR4 sequence and the gene was amplified from S. pneumoniae serotype 3 strain WU2. The amplified gene was inserted into the pHAT vector as described in Example 3.

Thirty-nine percent of rGtS-immunized mice survived a lethal bacterial challenge, whereas no control mice survived. The results suggested that GtS, an age-dependent S. pneumoniae antigen, is capable of inducing a partially protective immune response against S. pneumoniae in mice.

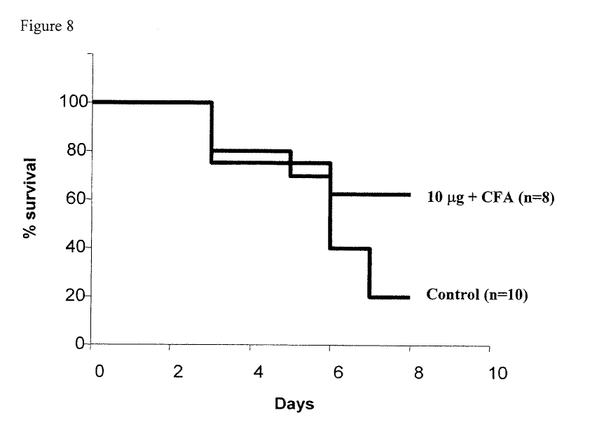

Active immunization with rGtS using CFA as adjuvant: BALB/c mice were immunized three times IM with 10 μg of rGtS in CFA/IFA/IFA in 3 weeks intervals. Mice were subsequently challenged with S. pneumoniae serotype 3 strain WU2. Survival was monitored up to 8 days after challenge. As depicted in FIG. 8, sixty percent of immunized mice survived the intranasal lethal challenge as opposed to 20% of adjuvant immunized (control) mice.

### EXAMPLE 8

Active Immunization with NADH Oxidase (NOX)

The cloning of the gene was by amplification of the gene using primers constructed according to the R6 sequence and the gene was amplified from S. pneumoniae R6. The amplified gene was inserted into the pHAT vector as described in Example 3.

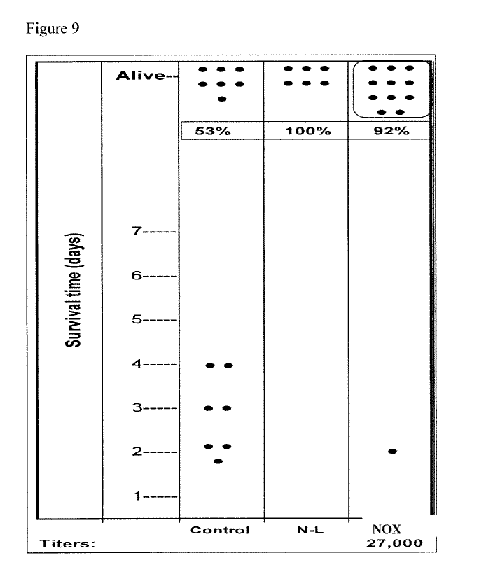

BALB/c mice were IP immunized with 25 μg of rNOX protein (NP_{—}345923, SEQ ID NO:8), 10 μg of a mixture of non-lectin (NL) proteins as a positive control and adjuvant only as a negative control. The immunizations were performed in the presence of CFA in the first immunization and IFA in the following 2 booster immunizations given in two weeks intervals. Mice were subsequently challenged with a lethal dose of S. pneumoniae serotype 3 strain (WU2). Survival was monitored daily for 7 days. While only 50% of control mice survived the bacterial challenge 100% of NL immunized and 92% of rNOX immunized mice survived the challenge as shown in FIG. 9.

### EXAMPLE 9

Passive Immunization with Pneumococcal Surface Immunogenic Protein B (PsipB; NP358083, SEQ ID NO:34).

The cloning of the gene was by amplification of the gene using primers constructed according to the TIGR4 sequence and the gene was amplified from S. pneumoniae serotype 3 strain WU2. The amplified gene was inserted into the pHAT vector as described in Example 3.

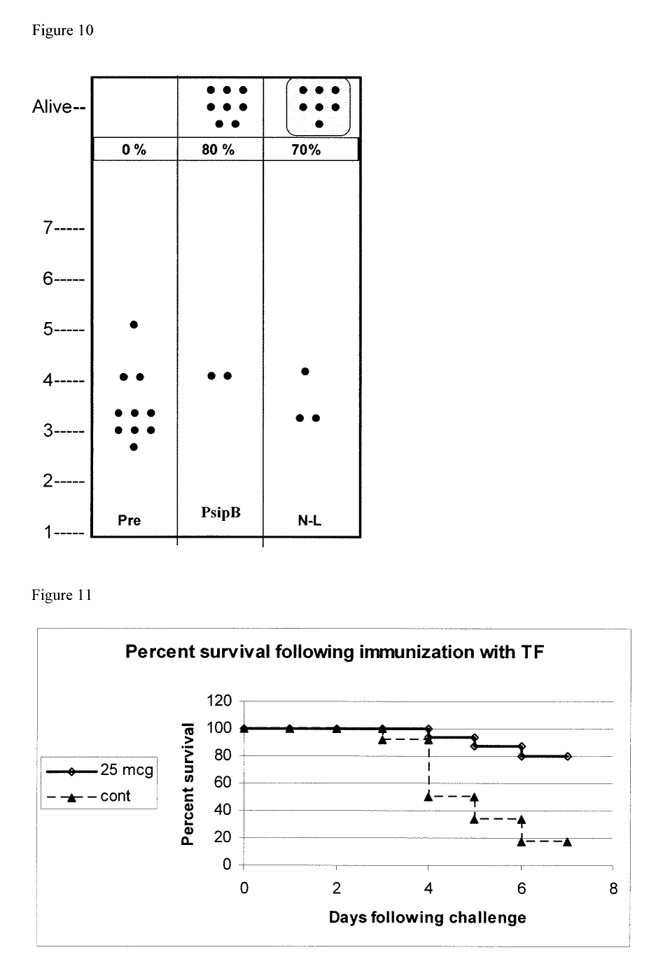

BALB/c mice were IP passively immunized two times with 100 μl of anti-PsipB antiserum 24 and 3 hours prior to bacterial challenge. Mice were IP challenged with S. pneumoniae strain 3 (WU2). Survival was monitored up to 7 days. Administration of either anti PsipB antiserum or the anti NL antisera protected the mice (80 and 70% respectively, FIG. 10) from a lethal challenge, while the control (preimmune) serum did not protect the mice from such challenge.

### EXAMPLE 10

Active Immunization with Trigger Factor (TF, NP 344923, SEQ ID NO:6).

The cloning of the gene was by amplification of the gene using primers constructed according to the TIGR4 sequence and the gene was amplified from S. pneumoniae strain R6. The amplified gene was inserted into the pET32a+ vector lacking the thioredoxin sequence. The vector contain a 5.7kDs tag protein which contains 6 consecutive histidines.

BALB/c mice were IP immunized (three times; CFA/IFA/IFA) with 25 μg of TF. Mice were subsequently challenged IN with S. pneumoniae serotype 3 strain WU2. Survival was monitored for 21 days. 25 μg TF elicited a protective immune response against a lethal challenge (80%) while mice immunized with adjuvant only were not protected (19% and 23 survival, respectively, FIG. 11)

### EXAMPLE 11

### FtsZ Cell Division Protein (NP_{—}346105, SEQ ID NO:10)

The cloning of the gene was by amplification of the gene using primers constructed according to the TIGR4 sequence and the gene was amplified from S. pneumoniae strain R6. The amplified gene was inserted into the pET32a+ vector lacking the thioredoxin sequence. The vector contain a 5.7kDs tag protein which contains 6 consecutive histidines.

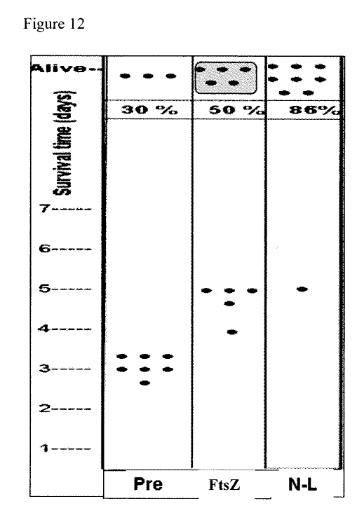

BALB/c mice were IP challenged with S. pneumoniae serotype 3 strain WU2 after 1 hour neutralization with rabbit anti-FtsZ antiserum, preimmune serum or anti NL serum. Survival was followed up to 7 days. Both the anti FtsZ and the anti NL antisera protected the mice from a lethal challenge (50% and 86%, respectively), while the preimmune serum protected 30% of the challenged mice (FIG. 12).

### EXAMPLE 12

### PTS System, Mannose-specific IIAB Components NP_{—}344822, SEQ ID NO:25)

The cloning of the gene was by amplification of the gene using primers constructed according to the TIGR4 sequence and the gene was amplified from S. pneumoniae strain R6. The amplified gene was inserted into the pET32a+ vector lacking the thioredoxin sequence. The vector contain a 5.7kDs tag protein which contains 6 consecutive histidines.

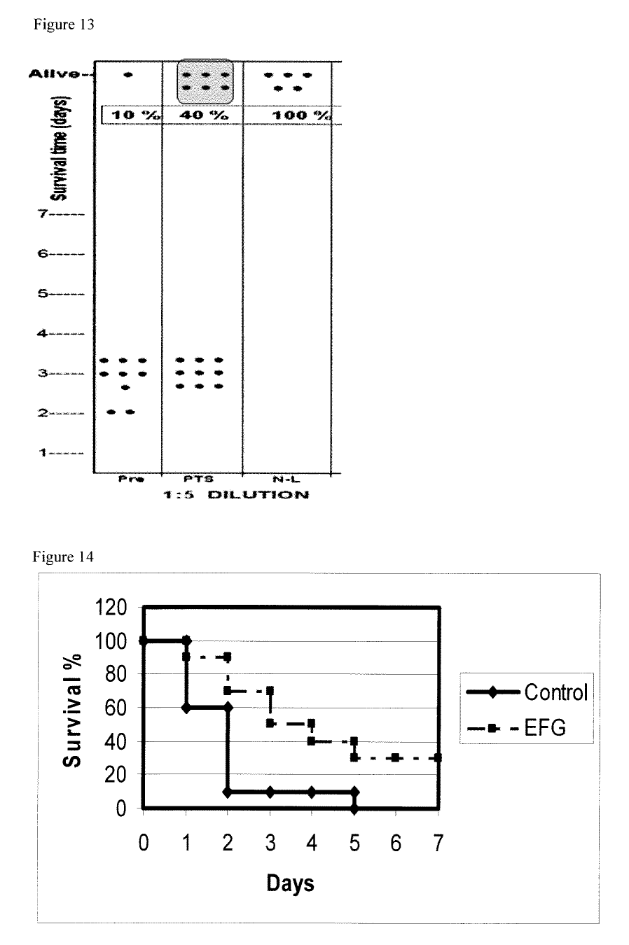

BALB/c mice were IP challenged with S. pneumoniae strain 3(WU2) after 1 hour neutralization with rabbit anti-PTS antiserum. Survival was followed up to 7 days. Both the anti-PTS and the anti NL antisera protected the mice from a lethal challenge (40 and 100%, respectively), while only 10% of mice survived following challenge with bacteria pretreated with preimmune serum (FIG. 13).

### EXAMPLE 13

Vaccination with 6-phosphogluconate Dehydrogenase (6PGD, NP357929, SEQ ID NO:22)

Use of 6PGD for inducing protective immune response in mice was described in Daniely et al., 144:254-63. 2006. Immunization of mice with r6PGD protected 60% of mice for 5 days and 40% of the mice for 21 days following intranasal lethal challenge, while non of the control mice survived the same challenge after four days.

### EXAMPLE 14

Active Immunization with Elongation Factor G (EFG, NP344811, SEQ ID NO:7)

The cloning of the gene was by amplification of the gene using primers constructed according to the R6 sequence and the gene was amplified from S. pneumoniae strain S. pneumoniae serotype 3 strain WU2. The amplified gene was inserted into the pHAT vector lacking the thioredoxin sequence. The vector contains a 5.7kDs tag protein which contains 6 consecutive histidines.

BALB/c mice were immunized IP with 25μg of EFG in the presence of Alum. Mice were subsequently challenged IN with S. pneumoniae serotype 3 strain WU2. Survival was monitored for 21 days. As shown in FIG. 14, EFG elicited a protective immune response against a lethal challenge in 30% of the mice, while all control mice, immunized with adjuvant only, succumbed 5 days following the bacterial challenge.

### EXAMPLE 15

### Clinical Studies

The first Phase 1 study is performed in 20-25 adults, testing the candidate vaccine for safety and immunogenicity. The second Phase 1 study evaluates 2 or 3 dosage levels of the vaccine in groups of 20-25 infants each for safety and immunogenicity.

The first Phase 2 study is performed in 100-150 infants at a developed world site using the dosage level chosen in Phase 1, and evaluates safety and immunogenicity as well as obtain more information about a potential surrogate assay. The second Phase 2 study at a developed world site is performed in 300-500 in infants in multiple sites, and evaluates interactions with other concomitant vaccines for extended safety and immunogenicity. The third Phase 2 study is performed in parallel 200 infants at the developing world location at which the Phase 3 efficacy study performed, to confirm immunogenicity and safety before Phase 3.

The Phase 3 efficacy study would be performed in a developing world site in 50,000 infants as a placebo-controlled double-blind study with a clinical endpoint.

The Phase 3 immunogenicity study would be performed in parallel in a developed world site using 3 different lots of final manufacturing-scale vaccine in 4 groups of 200 infants each. The Phase 3 safety study would be performed in parallel in 10,000 infants in developed world sites.

### EXAMPLE 16

### Verification of Immunogenicity and Age-dependency of Nox and GtS

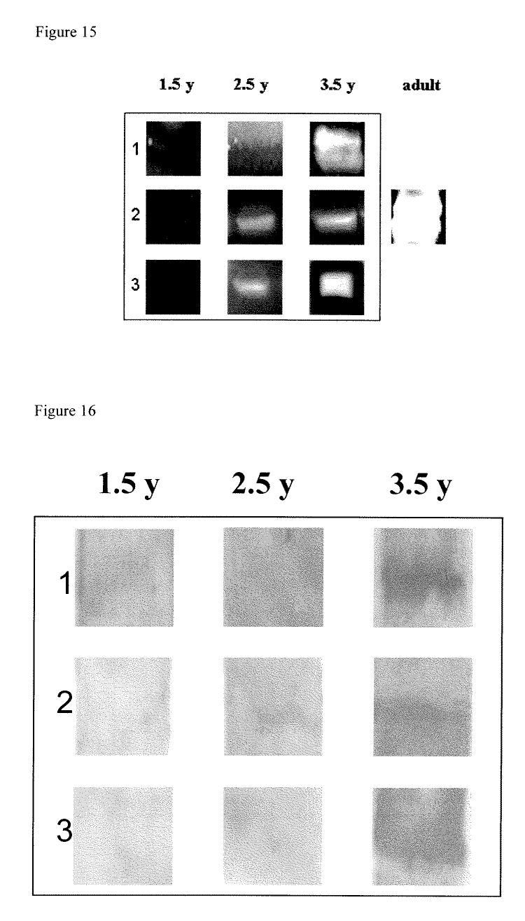

To verify that GtS induces an age-dependent immune response, sera from 3 healthy children attending day care centers (with documented episodes of carriage of different S. pneumoniae serotypes) were obtained longitudinally between 18-42 months of age. A representative series revealing quantitative and qualitative enhancement of antibody responses to rGtS protein over time is shown in FIG. 15. The rGtS protein was undetected by the infants' sera at 18 and slightly detected at 30 months of age. Maximal detection of rGtS with the children's sera was observed at 42 months of age. Sera obtained from a healthy adult detected rGtS to the highest extent.

Immunoblot analysis of rNOX with sera obtained longitudinally from children attending day-care centers demonstrated age-dependent enhancement in protein recognition in all 3 children (FIG. 16).

While specific embodiments of the invention have been described for the purpose of illustration, it will be understood that the invention may be carried out in practice by skilled persons with many modifications, variations and adaptations, without departing from its spirit or exceeding the bounds of the present invention.

What is claimed is:

1. A vaccine composition comprising as the active ingredient one or more isolated proteins selected from S. pneumoniae cell wall or cell membrane proteins or immunogenically-active fragments, derivatives or modifications thereof which are associated with an age-dependent immune response, optionally together with one or more pharmaceutically acceptable adjuvants.

2. The vaccine composition according to claim 1, wherein said S. pneumoniae cell wall and/or cell membrane protein is selected from the group consisting of: phosphoenolpyruvate protein phosphotransferase (Accession No. NP_{—}345645, SEQ ID NO:4); phosphoglucomutase/phosphomannomutase family protein (Accession No. NP_{—}346006, SEQ ID NO:5); trigger factor (Accession No. NP_{—}344923, SEQ ID NO:6); elongation factor G/tetracycline resistance protein (tetO), (Accession No. NP_{—}344811, SEQ ID NO:7); NADH oxidase (Accession No. NP_{—}345923, SEQ ID NO:8); Aspartyl/glutamyl-tRNA amidotransferase subunit C (Accession No. NP_{—}344960, SEQ ID NO:9); cell division protein FtsZ (Accession No. NP_{—}346105, SEQ ID NO: 10); L-lactate dehydrogenase (Accession No. NP_{—}345686, SEQ ID NO:11); glyceraldehyde 3-phosphate dehydrogenase (GAPDH), (Accession No. NP_{—}346439, SEQ ID NO:12); fructose-bisphosphate aldolase (Accession No. NP_{—}345117, SEQ ID NO:13); UDP-glucose 4-epimerase (Accession No. NP_{—}346261, SEQ ID NO:14); elongation factor Tu family protein (Accession No. NP_{—}358192, SEQ ID NO:15); Bifunctional GMP synthase/glutamine amidotransferase protein (Accession No. NP_{—}345899, SEQ ID NO:16); glutamyl-tRNA synthetase (Accession No. NP_{—}346492, SEQ ID NO:17); glutamate dehydrogenase (Accession No. NP_{—}345769, SEQ ID NO:18); Elongation factor TS (Accession No. NP_{—}346622, SEQ ID NO:19); phosphoglycerate kinase (TIGR4) (Accession No. AAK74657, SEQ ID NO:20); 30S ribosomal protein S1 (Accession No. NP_{—}345350, SEQ ID NO:21); 6-phosphogluconate dehydrogenase (Accession No. NP_{—}357929, SEQ ID NO:22); aminopeptidase C (Accession No. NP_{—}344819, SEQ ID NO:23); carbamoyl-phosphate synthase (large subunit) (Accession No. NP_{—}345739, SEQ ID NO:24); PTS system, mannose-specific IIAB components (Accession No. NP_{—}344822, SEQ ID NO:25); 30S ribosomal protein S2 (Accession No. NP_{—}346623, SEQ ID NO:26); dihydroorotate dehydrogenase 1B (Accession No. NP_{—}358460, SEQ ID NO:27); aspartate carbamoyltransferase catalytic subunit (Accession No. NP_{—}345741, SEQ ID NO:28); elongation factor Tu (Accession No. NP_{—}345941, SEQ ID NO:29); Pneumococcal surface immunogenic protein A (PsipA) (Accession No. NP_{—}344634, SEQ ID NO:30); phosphoglycerate kinase (R6) (Accession No. NP_{—}358035, SEQ ID NO:31); ABC transporter substrate-binding protein (Accession No. NP_{—}344690, SEQ ID NO:32); endopeptidase O (Accession No. NP_{—}346087, SEQ ID NO:33); Pneumococcal surface immunogenic protein B (PsipB) (Accession No. NP_{—}358083, SEQ ID NO:34); Pneumococcal surface immunogenic protein C (PsipC) (Accession No. NP_{—}345081, SEQ ID NO:35).

3. The vaccine composition according to claim 2, wherein said S. pneumoniae cell wall and/or cell membrane protein is selected from the group consisting of: fructose-bisphosphate aldolase (FBA, NP_{—}345117, SEQ ID NO:13), Phosphoenolpyruvate protein phosphotransferase (PPP, NP_{—}345645 (SEQ ID NO:4), Glutamyl tRNA synthetase (GtS, NP_{—}346492, SEQ ID NO:17), NADH oxidase (NOX, NP_{—}345923, SEQ ID NO:8), Pneumococcal surface immunogenic protein B (PsipB; NP_{—}358083, SEQ ID NO:34), trigger factor (TF, NP 344923, SEQ ID NO:6), FtsZ cell division protein (NP_{—}346105, SEQ ID NO:10), PTS system, mannose-specific IIAB components (PTS, NP_{—}344822, SEQ ID NO:25), and Elongation factor G (EFG, NP_{—}344811, SEQ ID NO:7).

4. The vaccine composition according to claim 1, wherein the one or more S. pneumoniae cell wall and/or cell membrane proteins are lectins and the composition is formulated for administration to an infant under two to four years of age, or for administration to an immunocompromised or elderly subject.

5. The vaccine composition according to claim 1, wherein the one or more S. pneumoniae cell wall and/or cell membrane proteins are non-lectins.

6. A vaccine composition comprising at least one polynucleotide sequence encoding a protein selected from one or more S. pneumoniae cell wall or cell membrane proteins or immunogenically-active protein fragments, derivatives or modifications thereof, which is associated with an age-dependent immune response, optionally together with one or more pharmaceutically acceptable adjuvant.

7. The vaccine composition of claim 6 further comprising at least one polynucleotide sequence encoding an adjuvant peptide or protein.

8. The vaccine composition of claim 6, wherein the polynucleotide sequence encodes a protein selected from glyceraldehyde 3-phosphate dehydrogenase (GAPDH), (Accession No. NP_{—}346439, SEQ ID NO:12); fructose-bisphosphate aldolase (Accession No. NP_{—}345117, SEQ ID NO:13).

9. A method for protecting a human subject against infection with S. pneumoniae by administering to said subject a vaccine composition comprising one or more S. pneumoniae cell wall or cell membrane proteins, immunogenically-active protein fragments, derivatives or modifications thereof, which are associated with an age-dependent immune response and which are administered in an amount effective to induce an immune response to S. pneumoniae to thus protect said subject from infection with S. pneumoniae.

10. The method according to claim 9, wherein the one or more proteins are effective in all age groups, including those age groups that do not produce anti-S. pneumoniae antibodies following inoculation with polysaccharide-based vaccines.

11. The method according to claim 9, wherein the subject is an infant under two to four years of age.

12. The method according to claim 9, wherein the subject is an immunocompromised or elderly subject.

13. The method of claim 9, wherein said S. pneumoniae cell wall and/or cell membrane protein is selected from the group consisting of: phosphoenolpyruvate protein phosphotransferase (Accession No. NP_{—}345645, SEQ ID NO:4); phosphoglucomutase/phosphomannomutase family protein (Accession No. NP_{—}346006, SEQ ID NO:5); trigger factor (Accession No. NP_{—}344923, SEQ ID NO:6); elongation factor G/tetracycline resistance protein (tetO), (Accession No. NP_{—}344811, SEQ ID NO:7); NADH oxidase (Accession No. NP_{—}345923, SEQ ID NO:8); Aspartyl/glutamyl-tRNA amidotransferase subunit C (Accession No. NP_{—}344960, SEQ ID NO:9); cell division protein FtsZ (Accession No. NP_{—}346105, SEQ ID NO:10); L-lactate dehydrogenase (Accession No. NP_{—}345686, SEQ ID NO:11); glyceraldehyde 3-phosphate dehydrogenase (GAPDH), (Accession No. NP_{—}346439, SEQ ID NO:12); fructose-bisphosphate aldolase (Accession No. NP_{—}345117, SEQ ID NO:13); UDP-glucose 4-epimerase (Accession No. NP_{—}346261, SEQ ID NO:14); elongation factor Tu family protein (Accession No. NP_{—}358192, SEQ ID NO:15); Bifunctional GMP synthase/glutamine amidotransferase protein (Accession No. NP_{—}345899, SEQ ID NO:16); glutamyl-tRNA synthetase (Accession No. NP_{—}346492, SEQ ID NO:17); glutamate dehydrogenase (Accession No. NP_{—}345769, SEQ ID NO:18); Elongation factor TS (Accession No. NP_{—}346622, SEQ ID NO:19); phosphoglycerate kinase (TIGR4) (Accession No. AAK74657, SEQ ID NO:20); 30S ribosomal protein S1 (Accession No. NP_{—}345350, SEQ ID NO:21); 6-phosphogluconate dehydrogenase (Accession No. NP_{—}357929, SEQ ID NO:22); aminopeptidase C (Accession No. NP_{—}344819, SEQ ID NO:23); carbamoyl-phosphate synthase (large subunit) (Accession No. NP_{—}345739, SEQ ID NO:24); PTS system, mannose-specific IIAB components (Accession No. NP_{—}344822, SEQ ID NO:25); 30S ribosomal protein S2 (Accession No. NP_{—}346623, SEQ ID NO:26); dihydroorotate dehydrogenase IB (Accession No. NP_{—}358460, SEQ ID NO:27); aspartate carbamoyltransferase catalytic subunit (Accession No. NP_{—}345741, SEQ ID NO:28); elongation factor Tu (Accession No. NP_{—}345941, SEQ ID NO:29); Pneumococcal surface immunogenic protein A (PsipA) (Accession No. NP 344634, SEQ ID NO:30); phosphoglycerate kinase (R6) (Accession No. NP_{—}358035, SEQ ID NO:31); ABC transporter substrate-binding protein (Accession No. NP_{—}344690, SEQ ID NO:32); endopeptidase O (Accession No. NP_{—}346087, SEQ ID NO:33); Pneumococcal surface immunogenic protein B (PsipB) (Accession No. NP_{—}358083, SEQ ID NO:34); Pneumococcal surface immunogenic protein C (PsipC) (Accession No. NP_{—}345081, SEQ ID NO:35).

14. The method of claim 13, wherein said S. pneumoniae cell wall and/or cell membrane protein is selected from the group consisting of: fructose-bisphosphate aldolase (FBA, NP_{—}345117, SEQ ID NO:13), Phosphoenolpyruvate protein phosphotransferase (PPP) NP_{—}345645 (SEQ ID NO:4), Glutamyl tRNA synthetase (GtS, NP_{—}346492, SEQ ID NO:17), NADH oxidase (NOX, NP_{—}345923, SEQ ID NO:8), Pneumococcal surface immunogenic protein B (PsipB; NP_{—}358083, SEQ ID NO:34), trigger factor (TF, NP 344923, SEQ ID NO:6), FtsZ cell division protein (NP_{—}346105, SEQ ID NO:10), PTS system, mannose-specific IIAB components (PTS, NP_{—}344822, SEQ ID NO:25), and Elongation factor G (EFG, NP344811, SEQ ID NO:7).

15. The method according to claim 9, wherein composition includes one or more pharmaceutically acceptable adjuvants.

16. The method according to claim 9, wherein the one or more S. pneumoniae proteins to be administered are lectins and wherein the immune response is effective against localized infection of a mucosal tissue by S. pneumoniae.

17. The method according to claim 9, wherein the one or more S. pneumoniae proteins to be administered are non-lectins and wherein the immune response is effective against systemic infection with S. pneumoniae.

18. The method according to claim 9, wherein the subject is protected against S. pneumoniae infection by administration of the vaccine composition prior to occurrence of said infection.

19. A method for identifying proteins having age-dependent immunogenicity against pathogens expressing said proteins comprising the steps of: providing an extract of the cell wall and/or cell membrane of the pathogen; separating the extract by 2D-electrophoresis or micro-chromatography; blotting the protein extract to a matrix; probing the blots with sera collected longitudinally from children at different ages; identifying the protein spots having intensity increasing with; thereby identifying a protein having age-dependent immunogenicity.

20. The method of claim 19, wherein the pathogen is S. pneumoniae or Streptococcus pyogenes.

21. A vaccine compositions comprising at least one protein identified by the method of claim 19.
